(19) United States
(12) Patent Application Publication
(10) Pub. No.: US 2021/0316144 A1
(43) Pub. Date: Oct. 14, 2021

(54) Systems Methods And Devices For Closed-Loop Stimulation To Enhance Stroke Recovery

(71) Applicant: The Regents of the University of California, Oakland, CA (US)

(72) Inventors: Karunesh Ganguly, San Francisco, CA (US); Tanuj Gulati, San Francisco, CA (US); Dhakshin S. Ramanathan, San Diego, CA (US)

(21) Appl. No.: 17/259,760

(22) PCT Filed: Jul. 19, 2019
(86) PCT No.: PCT/US19/42617

§ 371 Date: Jan. 12, 2021

Related U.S. Application Data
(60) Provisional application No. 62/700,609, filed on Jul. 19, 2018.

(51) Int. Cl. A61N 1/36; A61N 1/05; A61B 5/313; A61B 5/00

(52) U.S. Cl. CPC A61N 1/36103; A61N 1/36171; A61B 5/4836; A61B 5/313; A61N 1/0531

(57) ABSTRACT

Systems, methods and devices for promoting recovery from a stroke induced loss of motor function in a subject. In certain aspects, the system includes at least one electrode, and an operations system in electrical communication with at least one electrode, wherein the at least one electrode is constructed and arranged to apply current across the brain of the subject and to record low frequency oscillations from a perilesional region of the subject. In certain aspects, provided is a method comprising placing at least one recording electrode in electrical communication in a perilesional region of the subject; placing at least one stimulation electrode in electrical communication with the brain of the subject; recording low frequency oscillations from the perilesional region of the subject; and delivering current stimulation to the brain of the subject.

### CROSS-REFERENCE TO RELATED APPLICATION(S)

This application claims priority to International PCT Application No. PCT/US19/42617, filed on Jul. 19, 2019, which claims the benefit of U.S. Provisional Application No. 62/700,609, filed on Jul. 19, 2018; which is incorporated herein by reference in its entirety.

### GOVERNMENT SUPPORT

This invention was made with government support under VA Merit 1I01RX001640 awarded by the Veterans Health Administration and 1K02NS093014 from NINDS/NIH and R01MH111871 from NIMH/NIH. The government has certain rights in the invention.

### BACKGROUND OF THE INVENTION

Stroke is the leading cause of motor disability in the United States, affecting over 700,000 patients each year. No pharmacological or mechanical therapies are currently approved to enhance function during recovery from stroke. Intensive physical therapy to help relearn and regain impaired motor functions is the only currently available treatment for stroke patients and often is a slow and incomplete process.

The development of technologies to promote motor rehabilitation after stroke would be very beneficial. From a network perspective, the motor system is a complex organization of interconnected nodes. This highly dynamic system is capable of generating finely coordinated actions as well as adapting to damage to the network. However, the electrophysiological correlates of the recovery process are poorly understood. For example, it remains unclear what electrophysiological patterns predict either recovery or the lack of recovery. Moreover, it remains unclear how to precisely modulate the motor network in order to improve function after injury.

Some neuromodulatory techniques (both invasive and non-invasive) have been studied for the purpose of promoting motor learning and stroke recovery. In these neuromodulation therapies, an electric or chemical signal stimulates nerve cell activity. Such therapies include transcranial direct current stimulation (“tCS”), transcranial magnetic stimulation (“TMS”), epidural cortical stimulation (“ECS”), and peripheral nerve stimulation (“PNS”). However, the results have shown inconsistent or marginal improvements in recovery. Further, the majority of these studies—including the tCS and TMS therapies—use an ‘open-loop stimulation’ design in which the electric stimulation is continuously turned on for an extended time period of preprogrammed and constant stimulation that is uncoupled to behavior or ongoing brain activity and thus does not respond to patient movement or symptoms. This constant, unvarying stimulation can deliver too much or too little stimulus and is not adaptable to the specific patient needs.

There is a need in the art for neurostimulation devices, systems, and methods for effective treatment of stroke patients.

### BRIEF SUMMARY

Disclosed herein is a neurostimulation system for promoting subject recovery from a brain lesion that includes at least one electrode, and an operations system in electrical communication with at least one electrode, wherein the at least one electrode is constructed and arranged to apply current across the brain of the subject and to record low frequency oscillations from a perilesional region of the subject.

One Example relates to a method for promoting recovery from a stroke induced loss of motor function in a subject including placing at least one recording electrode in electrical communication in a perilesional region of the subject, placing at least one stimulation electrode in electrical communication with the brain of the subject, recording low frequency oscillations (LFOs) from the perilesional region of the subject, and delivering alternating current stimulation to the brain of the subject.

Implementations may include one or more of the following features. The method where the alternating current has a waveform selected from the group including of monopolar, biphasic, sinusoidal, and customized shapes created using decay and growth time constants. The method further including instructing the subject to perform a motor task and monitoring the performance of the subject on the motor task. The method further including increasing the amplitude of the delivered alternating current incrementally to the subject until a change in performance of the motor task is detected. The method further including decreasing the amplitude of the alternating current delivered to the subject following the detection of the change in motor task performance. The method where current is delivered to the perilesional region of the subject. The method where the alternating current is delivered to a sleeping subject. The method where the at least one stimulation electrode is disposed for synchronized cortical and subcortical stimulation. The method where the alternating current stimulation is delivered in phase with the recorded LFOs. The method where the alternating current stimulation is delivered at between about 0.1 and about 1000 Hz. The method where the alternating current stimulation is delivered in response to recorded electrical activity. The method where the alternating current stimulation is delivered in response to subject movement. The method where the one or more stimulation electrodes is placed in at least one of the subcortical white matter, basal ganglia, brainstem, cerebellum or thalamus of the subject. The method where the one or more stimulation electrodes is placed in at least one cortical area. The method where a second stimulation electrode is placed in at least one cortical area. The method where the cortical area the one or more stimulation electrode is placed in a cortical area of the subject selected from the group including of: perilesional, premotor-central (PMv), premotor-dorsal (PMd), supplementary motor area (SMA), supramarginal gyrus, parietal motor and sensory areas. The method where a second stimulation electrode is placed in at least one of the subcortical white matter, basal ganglia, brainstem, cerebellum or thalamus of the subject. The method further including recording at least one additional frequency wave selected from the group including of beta waves, high-gamma waves, gamma waves, alpha waves, delta waves, theta waves and waves of more than 300 Hz and spiking activity as a means of decoding movement intention.

Another Example relates to a neurostimulation system for improving recovery in a subject with a brain lesion, the neurostimulation system including: an electrode constructed and arranged to record low frequency oscillations, and an operations system, where the electrode and operations system are constructed and arranged to: record muscle movement of the subject, and deliver current to the brain of the subject upon co-occurrence of perilesional low frequency oscillations and subject muscle movement. deliver current to the brain of the subject in response to low frequency oscillations in the brain. Implementations may include one or more of the following features. The neurostimulation system where the delivered current is alternating current. Implementations of the described techniques may include hardware, a method or process, or computer software on a computer-accessible medium.

### BRIEF DESCRIPTION OF THE DRAWINGS

FIG. 1 shows schematic representations of the systems and methods according to certain embodiments.

FIG. 2 shows data indicating changes in Low-Frequency Oscillatory (LFO) dynamics during motor learning.

FIG. 3 shows data showing LFO dynamics during motor recovery after stroke, according to certain embodiments.

FIG. 4 shows modulation of LFO dynamics using direct current stimulation (CS), according to certain embodiments.

FIG. 5 shows data showing task-dependent CS improves motor function, according to certain embodiments.

FIG. 6 shows data indicating that precisely time-locked stimulation improves motor function.

FIG. 7 shows data showing enhancement of phase-locking with anodal TCS during sleep

FIG. 8 shows data showing movement-related low-frequency oscillations in sensorimotor cortex in humans.

FIG. 9 shows data showing low-frequency quasi-oscillatory (LFO) activity during a skilled forelimb reach task in healthy rats.

FIG. 10 shows data showing stroke diminished LFO activity in M1.

FIG. 11 shows data showing restoration of LFOs in perilesional motor cortex tracked motor recovery.

FIG. 12 shows data showing LFO activity increased with Direct Current Stimulation (DCS) in acute (anesthetized) recording sessions.

FIG. 13 shows data showing task-dependent DCS improved motor function post-stroke.

FIG. 14 shows localization of electrodes.

FIG. 15 shows data showing emerging control of skilled fine and gross movements is dissociable.

FIG. 16 shows data related to precise movement timing in skilled gross movements.

FIG. 17 shows data showing coordinated low-frequency activity across M1 and DLS representing control of skilled gross movements.

FIG. 18 shows percentage of units displaying quasi-oscillatory activity increases during reach-to-grasp skill learning.

FIG. 19 shows percentage of units displaying quasi-oscillatory activity increases during reach-to-grasp skill learning.

FIG. 20 shows coordinated M1 and DLS activity is specifically linked to skilled gross, but not fine movements.

FIG. 21 shows that inactivation of DLS abolishes low-frequency M1 activity and disrupts skilled gross movements.

FIG. 22 shows the difference in reach amplitude for successful and unsuccessful trials before and after DLS inactivation.

FIG. 23 shows control of skilled fine movements is represented in M1.

FIG. 24 shows changes in GPFA neural trajectory consistency from day one to day eight.

FIG. 25 shows the application of ACS in animals.

FIG. 26 shows the natural variation of natural LFOs in motor cortex.

### DETAILED DESCRIPTION

Recent work has highlighted the importance of transient low-frequency oscillatory (LFO, <4 Hz) activity in the healthy motor cortex (M1) during skilled upper-limb tasks. These brief bouts of oscillatory activity may establish the timing or sequencing of motor actions. Here we show that LFOs track motor recovery post-stroke and can be a physiological target for neuromodulation. In rodents, we found that reach-related LFOs, as measured in both the LFP and related spiking activity, were diminished after stroke and that spontaneous recovery was closely correlated with their restoration in perilesional cortex. Sensorimotor LFOs were also diminished in a human subject with chronic disability after stroke in contrast to two non-stroke subjects who demonstrated robust LFOs. Therapeutic delivery of electrical stimulation time-locked to the expected onset of LFOs was found to significantly improve skilled reaching in stroke animals. Here we specifically claim that LFOs that are time-locked to cortical and subcortical targets can be used to improve motor function. Together, our results suggest that restoration or modulation of cortical oscillatory dynamics is important for recovery of upper-limb function and that they may serve as a novel target for clinical neuromodulation.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g., an animal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed systems and methods, the subject has been diagnosed with a need for treatment of one or more stroke related loss of motor function prior to the treatment step.

Certain implementations disclosed and contemplated herein relate to neurostimulation devices—and related systems and methods—that can detect low frequency oscillations in stroke patients and utilize that information to make treatment decisions. Further embodiments relate to neurostimulation devices, systems, and methods that can augment the low frequency oscillations (LFOs) by applying direct current to the patient, including, in some such embodiments, real-time application of direct current and/or responsive application of direct current in response to detection of predetermined oscillation levels. Such responsive embodiments could be responsive to patient brain waves and requests for task-directed movement.

In certain aspects, disclosed are a method, system and associated devices for improving the motor function of a subject having suffered a loss of motor function as the result of a stroke. In certain implementations, the method involves recording activity from perilesional regions of the subject's brain. Through the recording of perilesional activity, the method seeks to detect LFOs, which have been surprisingly found to correspond to motor task learning/relearning during recovery. In certain implementations, the method further involves the application of discrete pulses of CS to perilesional regions which has been surprisingly found to potentiate motor task related LFOs, which thereby enhances relearning and recovery of motor function.

In certain embodiments, the application of CS is triggered by the detection of perilesional LFOs. In certain alternative embodiments, the application of CS is triggered by the onset of the subject's attempt to perform a motor task. In these embodiments, the CS may be delivered concurrently with the onset of the task attempt or immediately preceding task attempt. In still further alternative embodiments, CS is triggered by the co-occurrence of LFO detection and task attempt.

Disclosed herein is a neurostimulation system for promoting subject recovery from a brain lesion that includes at least one electrode, and an operations system in electrical communication with at least one electrode, wherein the at least one electrode is constructed and arranged to apply current across the brain of the subject and to record low frequency oscillations from a perilesional region of the subject.

In certain aspects, the at least one electrode is a single electrode capable of both recording LFOs and delivering current to the subject. In further embodiments, the at least one electrode comprises at least one recording electrode and at least one stimulation electrode for delivery of current to the brain of the subject. In certain aspects, electrodes are cranial screws. In further embodiments, the electrodes are one or more subdural electrodes. In exemplary embodiments, the one or more subdural electrodes comprise a plurality of electrodes arranged in an array. In these embodiments, the electrodes may be placed on a perilesional region of the motor cortex. According to still further embodiments, the one or more electrodes are depth electrodes, placed in one or more subcortical structure.

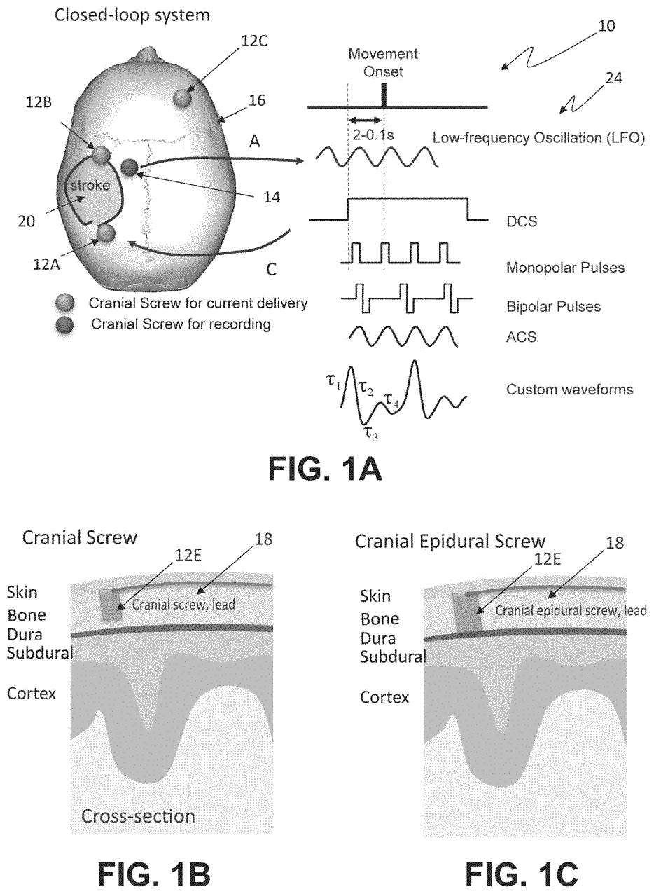

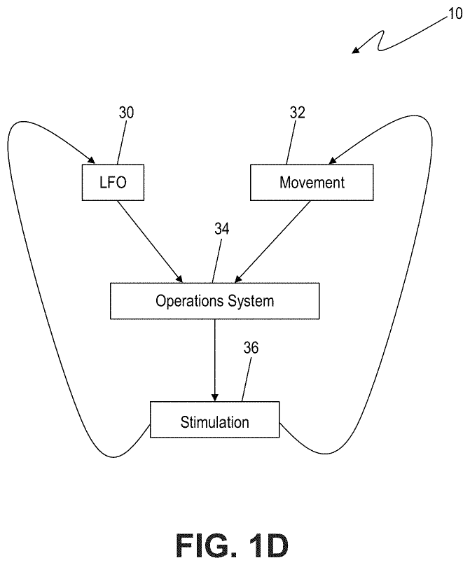

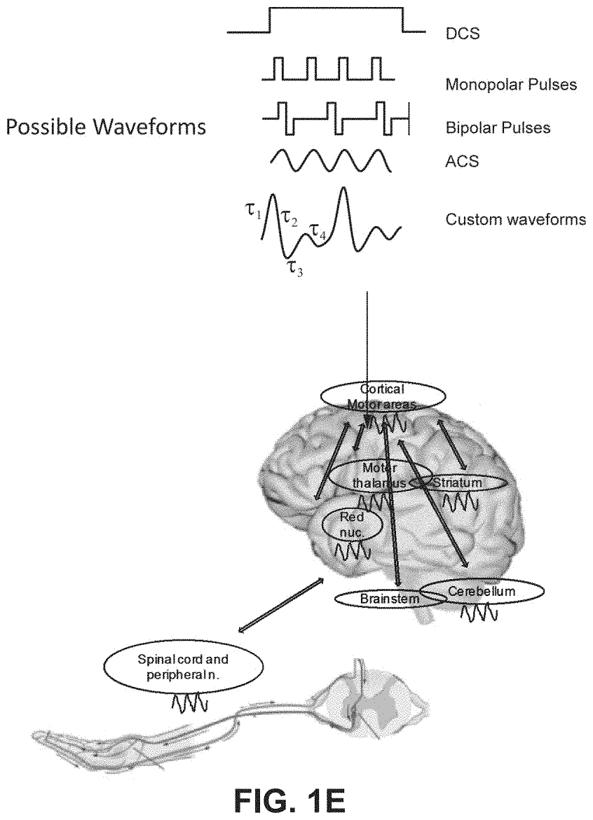

In certain aspects, the current delivered by the system is direct current stimulation. According to certain alternative embodiments, the current stimulation delivered by the system is alternating current stimulation. In exemplary aspects of these embodiments, the operations system delivers alternating current stimulation in phase with the recorded low frequency oscillations. In further embodiments, the alternating current stimulation (ACS) is delivered at a predetermined frequency. For example, in certain embodiments, the ACS is delivered at between about 0.1 to about 1000 Hz. In further embodiments, the ACS is delivered at between about 0.1 to about 4 Hz. In certain implementations, the ACS is delivered at about 3 Hz. In certain embodiments, the frequencies may be dynamically altered during the course of stimulation. For example, customized waveforms can be created using a sequence of exponential increase and decay series with a selected range of time constants. For example, in FIG. 1A, a customized waveform is made with 4 such functions. It is possible to use an arbitrary set of such exponential functions to create customized waveforms.

In certain aspects, the operations system is constructed and arranged to apply AC or DC current in response to recorded electrical activity. According to alternative embodiments, the operations system is constructed and arranged to deliver current in response to subject movement.

Disclosed herein is a method for promoting recovery from a stroke induced loss of motor function in a subject comprising placing at least one recording electrode in electrical communication in a perilesional region of the subject; placing at least one stimulation electrode in electrical communication with the brain of the subject; recording low frequency oscillations from the perilesional region of the subject; and delivering current stimulation to the brain of the subject.

In certain aspects of the instantly disclosed method, the current stimulation is delivered by direct current stimulation.

According to certain alternative embodiments, of the disclosed method, current stimulation is delivered by alternating current stimulation, delivered in phase with the low frequency oscillations. According to these embodiments, the LFO recorded at the perilesional site is used to determine the stimulation parameters of the alternating current stimulation. That is, the wave form and frequency of the alternating current stimulation is calculated to match the recorded LFO. In exemplary embodiments, the onset of the alternating current stimulation is concurrent with a peak of a low frequency oscillation waveform.

According to further aspects, the method further comprises the step of instructing the subject to perform a predefined motor task. In these embodiments, the motor task is predetermined to target the motor function effected by the brain lesion. In certain embodiments, current stimulation is delivered concurrently with subject's performance of the motor task. In further embodiments, the onset of the current stimulation immediately precedes instruction to the subject to perform the motor task. In exemplary embodiments, the onset of current stimulation is about 500 ms prior to the motor task and continues through the completion of the motor task. According to certain alternative embodiments, the current stimulation is triggered by the co-occurrence of motor task performance and LFO detection.

In certain aspects, the disclosed method is performed during sleep of the subject. In such embodiments, application of CS or ACS (0.1-1000 Hz) during sleep potentiate LFOs associated with recovery of motor function. In certain exemplary embodiments, during sleep following a training session, LFOs associated with improvement-related plasticity can be further potentiated by application of CS or ACS.

In certain aspects, current stimulation is delivered to the perilesional region of the subjects brain. According to certain alternative embodiments, the current is also delivered to one or more subcortical structures. Exemplary structures include but are not limited to the striatum, motor thalamus, red nucleus, cerebellum, red nucleus and/or spinal cord structures and peripheral structures. According to certain exemplary embodiments, alternating current stimulation is delivered to these structures, in phase with LFO recorded in the perilesional region during motor task performance.

Further disclosed herein is a neurostimulation system for improving recovery in a subject with a brain lesion, the neurostimulation system comprising: an electrode; and an operations system, wherein the electrode and operations system are constructed and arranged to deliver current to the brain of the subject in response to low frequency oscillations in the brain.

In certain aspects, the neurostimulation system, further comprises at least one electromyography electrode, constructed and arranged to record muscle movement of the subject. According to exemplary embodiments, the operations system delivers current to the brain of the subject upon co-occurrence of perilesional low frequency oscillations and subject muscle movement.

Turning now to the figures, FIG. 1A depicts an overview of the closed-loop stimulation (CLS) system 10 according to one implementation. In these implementations, the CLS system 10 is triggered by task-related low-frequency oscillation (LFO) power. As is shown in FIG. 1A, the system 10 has at least one electrode 12, 14, here a delivery electrode 12 and at least one recording electrode 14. In these implementations, the screws 12, 14 are implanted or otherwise disposed on the skull 16 of the patient. In various implementations, these electrodes 12, 14 are cranial screws, though other kinds of electrical, implantable devices are also contemplated.

As shown in FIGS. 1B-C, in certain implementations, the distal end 12A of an electrode or screw can be disposed partially through the skull bone 18 (FIG. 1B), such that there is no penetration of the cranial vault. In alternate implementations, the distal end 12E can be disposed through the skull bone 18 so as to be in the epidural space (FIG. 1C). It is understood that in further implementations the screws may be placed subdurally or even intracortically, such as disposing the distal end such that it is touching and/or penetrating the cortex itself. It is understood that further implementations and combinations of these placements are possible, such that the distal ends are disposed so as to best deliver and/or receive current in the desired application or implementation.

Returning to FIG. 1A, in various implementations, the various delivery electrodes 12A, 12B can be disposed perilesionally, adjacent to, or proximal to the lesion 20. Other delivery screws 12C can be disposed apart from the lesion 20, such as near the frontal cortex. The recording screw 14 can also be disposed perilesionally, adjacent to, or proximal to the lesion 20. In various implementations, both the delivery screws 12A, 12B, 12C and recording screws 14 are in electrical communication with an external operations system (generally at 24). The operations system 24 is configured to deliver current stimulation (CS) by way of the delivery screws (as is shown in relation to screw 12A) and receive low frequency oscillation signals (LFO) from the recording screw 14. In an alternate embodiment, both recording and stimulation can be achieved through the same cranial screws.

In various implementations, the operations system 24 is a closed-loop and is configured to apply CS and record LFO on a time-scale and compare it with recorded patient movement. In certain implementations, the movement of an area of the body will trigger LFO. In certain implementations, in response to observed LFO (reference arrow A), the operations system 24 can apply (reference arrow B) stimulation (reference arrow C) to the subject's brain through the delivery screws 12A, 12B.

As shown in FIG. 1A, in various implementations, the CS can be delivered, by direct current stimulation (DCS), alternating current stimulation (ACS), in monopolar pulses, bipolar pulses or other wave forms, as is also shown in FIG. 1E.

In use, and as is shown in FIG. 1D, in an exemplary implementation, electrodes 12, 14 are affixed to or otherwise disposed within the head of the patient 1. In these implementations, these electrodes are in electrical communication with an operations system 30 via wires or other connections. In various implementations, the operations system can be a desktop or handheld device constructed and arranged to send and receive electrical signals and/or currents. In various implementations, the operations system 34 has a processor, and can be any computer or processor known to those skilled in the art. In one embodiment, the operations system 34 includes software, which may be hosted in at least one or more computer servers, and can further comprise any type of known server, processor, or computer, any of which can run on a variety of platforms.

In accordance with one implementation, the operations system 34 has a central processing unit (“CPU”) and main memory, an input/output interface for communicating with various databases, files, programs, and networks (such as the Internet, for example), and one or more storage devices. The storage devices may be disk drive devices, CD ROM devices, or the cloud. The operations system 30 may also have an interface, including, for example, a monitor or other screen device and an input device, such as a keyboard, a mouse, a touchpad, or any other such known input device. Other embodiments include corresponding computer systems, apparatus, and computer programs recorded on one or more computer storage devices, each configured to perform the actions of the methods.

It is further understood that in certain implementations, such as that of FIG. 1D, the system 10 can also include one or more peripheral monitoring devices or systems 15. In various implementations, these peripheral monitoring systems 15 can be electrodiagnostic devices or systems such as electromyographs 15 or other known diagnostic or monitoring devices. It is further understood that the peripheral monitoring system 15 is not essential for operation of the system 10.

It is understood that in use according to various implementations, the system and various methods can be executed via a number of optional steps. In one step, and as shown in FIG. 1E, LFO from the recording electrode (shown as box 30 in FIG. 1E) and/or movement—such as electromyography (EMG) from the peripheral monitoring system 15 (box 32)—can be recorded by the operations system (box 34), which can also apply electrical stimulation (box 36). It is understood that in certain implementations, another step involves the application of current (box 36). In certain implementations, the application of current (box 36) can be initiated by detection of LFO (box 30). In further implementations, the application of current (box 36) can be initiated by movement of the subject (box 32). In further implementations, these steps can be performed concurrently, consecutively or independently.

In embodiments in which CS is triggered independently, a healthcare provider, such as a physical therapist, can trigger the application of CS as in conjunction with instructing the subject to perform a therapy related motor task. It is further understood that the onset of stimulation can include “pre-movement” stimulation, which can be titrated between seconds to milliseconds prior to movement. Alternate embodiments use different neural signatures for CLS. For example, a combination of LFO with EMG signals in proximal arm muscles (e.g. deltoid, trapezius or latisssimus dorsi) can trigger CS. In this implementation, the LFO and the EMG can be used equally to trigger CS. In further implementations, the EMG signal from proximal muscles could also be used alone to trigger the “pre-movement” CS. In yet further embodiments, movement is detected by sensors placed on the body of the subject. For example, one or more accelerometers can be placed on the limbs of the subject and signals from the one or more accelerometers can be used to trigger CS.

As described herein, in certain implementations the application of CS corresponds to task performance by the subject. That is, in certain implementations CS application is increased until the subject's performance on a task improves, and then the CS is reduced. This can be done in a closed-loop manner in which the parameters—frequency, waveform shape, amplitude and the like—are modulated in response to ongoing detected changes in behavior, such as finger movements, rate of movement and the like.

In certain implementations, ACS is utilized. In certain of these implementations, the ACS application is applied at about 0.3-4 Hz or at about a mean frequency of 3 Hz.

For the various biphasic waveform shapes, a longer ramp down than ramp up phase can be implemented, such that for example the ramp down ranges from two- to 100-fold slower than the ramp up. In certain implementations, the ramping down is 2.5× slower than the ramping up, for example 200 μs up phase duration, and 500 μs down phase duration. In these and other implementations, the application is charge balanced. In various implementations, the application of current ran range from about 1 μAmp to about 50 mA, that is, for very brief pulses within safe current density parameters, as would be understood.

In various implementations, the application of ACS is gated. For example, in certain implementations the ACS is applied in response to the initial onset of the LFO. In certain implementations, the threshold condition is met. In exemplary implementations, the threshold is the detection of a change in the LFO of a predetermined amount over the noise floor. In exemplary embodiments, that the predetermined threshold is about 2 or more standard deviations above the noise floor. If further implementations, the gating has more than one threshold.

In certain implementations, other pre-movement gating thresholds can be utilized alone or in combination with the detection of LFO onset, such as detected beta oscillations, including beta oscillations from about 10 Hz to about 40 Hz, including in combination with the onset of LFO or when the relationship between the beta oscillations and delta oscillations passes a defined ratio, such as <2.

In various implementations, the CS is directed at a signal target, while in alternate implementations multiple targets are used, as is shown in FIG. 1E. In certain implementations the target or targets include cortical targets; in further implementations striatal targets are utilized. Further implementations target deep areas while others are superficial.

In certain implementations the CS stimulation induces low frequency oscillations, such as synchronous low frequency oscillations across several neural areas or regions.

### EXPERIMENTAL EXAMPLES

The following examples are put forth so as to provide those of ordinary skill in the art with a complete disclosure and description of how the articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

It is commonly hypothesized that restoration of normal neural dynamics in the injured brain can improve function. However, we lack a precise neurophysiological framework for such an approach. Here we show that low-frequency oscillatory (LFO) dynamics play a critical role in the execution of skilled behaviors in both the intact and injured brain. We chronically recorded local field potentials and spiking during motor training in both healthy and post-stroke rats. Interestingly, we found that task-related LFOs emerged with skilled performance under both conditions and were a robust predictor of recovery. We further hypothesized that boosting LFOs might improve function in animals with persistent deficits. Strikingly, we found that direct current stimulation could boost LFOs, and when applied in a novel, task-dependent manner, significantly improved function in those with chronic deficits. Together, our results demonstrate that LFOs are essential for skilled controlled and represent a novel target for modulation after injury.

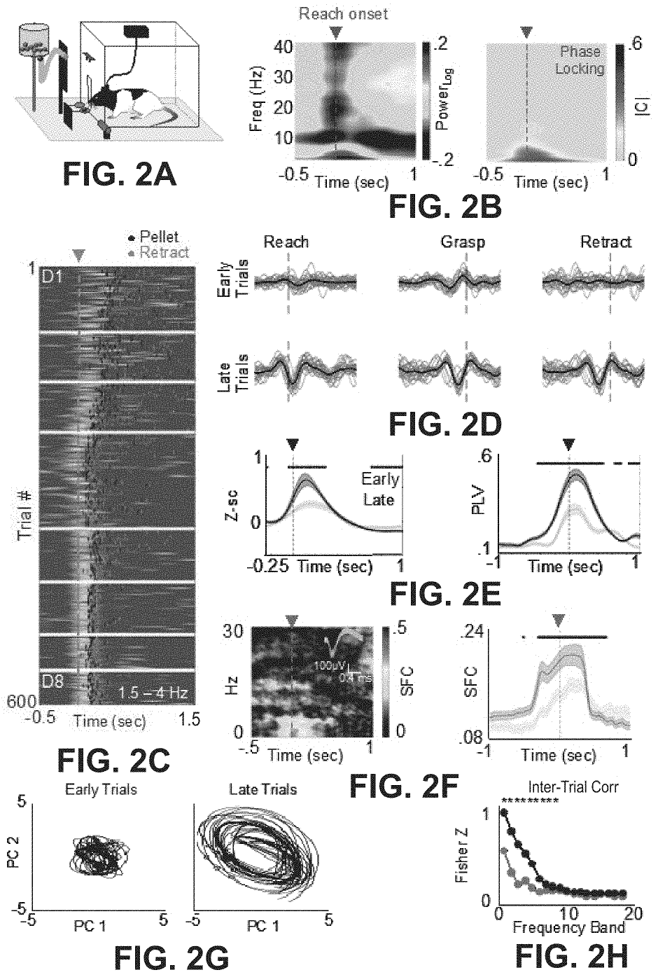

We first assessed the dominant LFP oscillatory dynamics associated with motor reaching in healthy animals, to confirm whether low-frequency oscillations, as identified in primates during motor actions, were similarly important in rodents. Rats were implanted with microwire arrays within motor cortex (M1) prior to learning a skilled forelimb reach task. This task required animals to reach out of a box, grasp a pellet placed on a small pedestal, and retract its arm back into the cage (FIG. 2A). This motor behavior requires significant dexterity of the distal forelimb and is dependent on M1. Animals were trained over multiple days using an automated reach-box that synced behavioral and electrophysiological data.

The dominant neural oscillation associated with the skilled motor reach task, i.e. averaged across all trials in all animals, occurred in the lowest-frequency bands (FIG. 2B). In addition to increased power, we also found significant task-related phase-locking of these lower-frequencies in association with the motor action. We next performed a t-test comparing changes in phase-locking at frequencies ranging from 1.5-60 Hz (a total of 117 frequencies), across a time-window from −10 ms to 500 ms compared to a pre-reach baseline period to assess which frequencies showed the largest evoked phase-locking related to reach-onset. We found that frequencies <8 and those between 11-13 Hz showed significant task-related phase-locking (paired t-test, p<0.05, FWE-corrected for 117 frequencies). However, frequencies <4 Hz showed the most significant phase-locking. Based on these results, we focused on the plasticity of low-frequency oscillations, i.e. <4 Hz, in motor leaning and recovery after stroke.

Interestingly, we found a clear evolution in LFOs with learning (FIG. 2C). As animals learned the task, the skilled motor action became temporally bound together and independent sub-movements became more phase-locked to the LFO (FIG. 2C-D), resulting in a significant increase in both task-related LFO power and LFO phase-locking to the motor actions; for this and all future calculations of LFO, unless otherwise indicated, we used frequencies <4 Hz (FIG. 2E, n=64 electrodes from 4 animals, *above is paired t-test p<0.05, FDR corrected). Averaged across a relevant reach-related time-window, i.e. −10 to 500 ms from reach-onset, we found on average an increase of 356±105% (p<0.01, paired t-test, n=64) in power and a 62±7.5% increase in phase-locking (p<0.001, paired t-test, n=64) with stable skill acquisition. It is important to note that trials were only included if the animal successfully reached and touched the pellet. We again performed a t-test comparing changes in phase-locking at frequencies from 1.5-60 Hz to assess which frequencies showed the largest change in phase-locking. Notably, we found that only frequencies <12 Hz showed an increase in phase-locking with learning; the largest changes occurred at frequencies <5 Hz; paired t-test, p<0.05, FWE-corrected).

It has been theorized that LFOs bind M1 microcircuits, including spiking activity of individual neurons, with mesoscale cortical dynamics (Bansal et al., 2011; Hall et al., 2014). We performed two types of analysis to probe this. We first assessed spike-field coherence (SFC), a measure of the relationship between spiking activity and the phase of oscillations at a specified frequency (Bokil et al., 2010; Buzsáki et al., 2012; Fries et al., 2001). We found many neurons demonstrated strong SFC to LFOs during the reach task, suggesting these slow oscillations play an important role in organizing M1 microcircuits (FIG. 2F). The strength of this coupling increased by 38±15%, as measured across the same time-window described above, when comparing SFC of neurons recorded on the first vs. the last day of skilled motor training (FIG. 2F n=62 from early and 64 from late sessions, 2-sample t-test, p<0.001).

To examine how motor training affects mesoscale LFO dynamics within M1, we used principle-components-analysis (PCA) to quantify dynamical patterns across M1 during task execution, using previously described techniques. Specifically, we plotted the trajectory of the first 3 principle-components, calculated across M1 channels, during the motor reach task. We found a striking increase in the stereotypy of these LFO neural trajectories with learning (FIG. 2G; shows examples of simultaneously plotted PC and movement trajectories from early and late trials in one animal). In order to quantify this effect across animals, we calculated the inter-trial correlation of the PC trajectory space across trials. The emergence of stereotyped mesoscale dynamical patterns occurred primarily in lower-frequency bands (maximal stereotypy increase with learning occurs at lower frequencies <5 Hz). FIG. 2G was calculated using the average Fisher-Z transformed inter-trial correlation value, n=4900 trials/frequency for early and late blocks, collected from 4 animals using 50 trials for each block. Stars indicate frequencies that show a significant increase in inter-trial correlation of the PC trajectory, using a p<0.001 threshold, FWE-corrected for 18 comparisons. Together, our results indicated that with skilled motor learning, the strength and phase-locking of LFOs increased and served to dynamically modulate the spiking activity of M1 neurons.

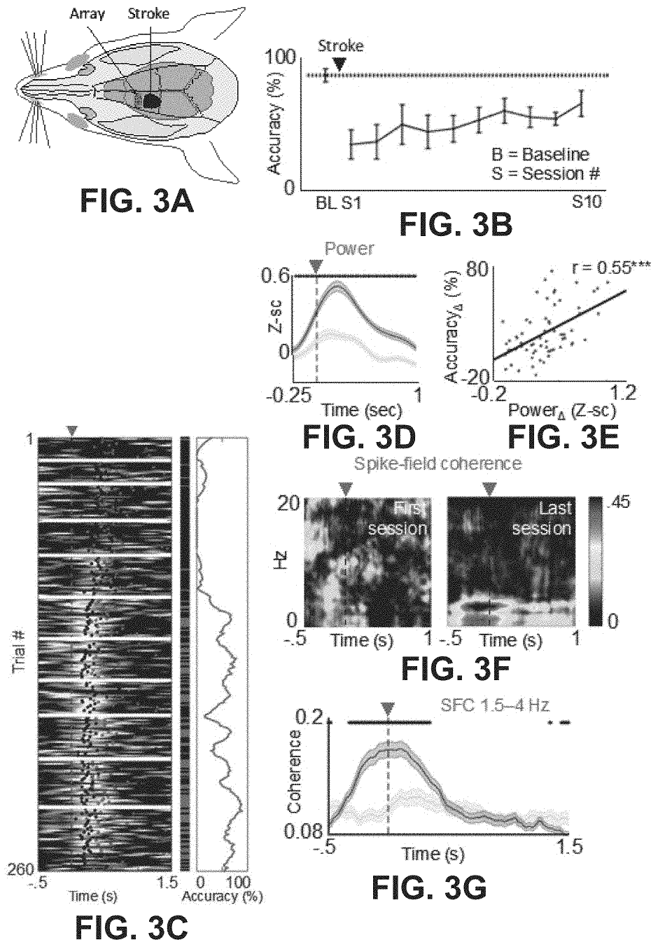

To probe the causal role of these cortical oscillations in the production of skilled motor behaviors, we used a photothrombotic stroke model to induce focal M1 lesions in well-trained animals. Immediately after the stroke, 16 or 32-channel micro-wire arrays were implanted in perilesional cortex anterior to the lesion, as described previously (FIG. 3A). Animals were given one week to recover from the stroke injury/electrode placement, after which they underwent motor training sessions on the same task for an additional 5-8 days to assess the relationship between motor recovery and modulation of task-related LFOs in perilesional cortex (FIG. 3B-C). Injury resulted in impaired motor performance; there was a drop in accuracy from 87±5% pre-lesion to 24±11% after the stroke, p<0.01. As expected, animals demonstrated an improvement in motor function over the course of training (FIG. 3B). The mean accuracy increased from 36±12% accuracy (average of first two sessions) to 65.4±7% (average of last two behavioral sessions), p<0.05, paired t-test.

Impaired motor performance after the injury was associated with diminished LFOs in perilesional cortex and recovery of motor function in each animal was linked with a strong increase in these LFOs (FIG. 3C). Over the course of rehabilitation training, animals demonstrated a 584±157% increase in LFO power (FIG. 3D, paired t-test of % increase in Z-scored power across channels averaged across reach-related time-window, n=176 channels from 6 animals, p<0.001). This increase in LFO power was a highly significant predictor of motor recovery. We specifically compared the mean LFO power change for a session (i.e. relative to the first session post-stroke across channels in each animal) and the average accuracy change (i.e. relative to the first session post-stroke; FIG. 3E, r=0.55, p<0.001). Consistent with the notion that LFOs dynamically organize motor cortical areas, we also found that recovery of function was associated with increased phase-locking of perilesional spiking activity with the low frequency oscillations (FIG. 3F example animal showing SFC for all units at two time points, FIG. 3G, n=127 units from early sessions, and 169 units from late sessions, p<0.001 2-sample t-test averaged across reach-related time-window). As with healthy animals, there was also a significant increase in task-related LFO phase-locking of 27±3.7%, p<0.001.

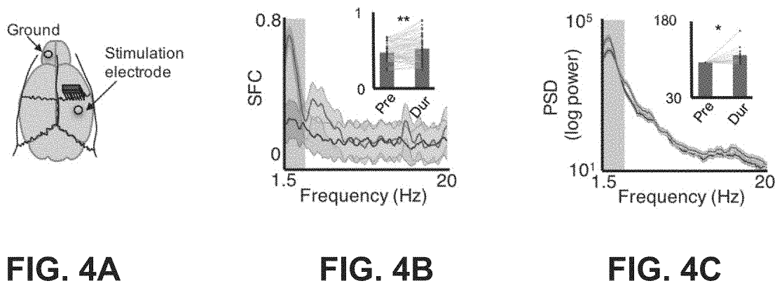

We next examined whether electrical stimulation targeted to LFOs might improve motor function after stroke. We analyzed the effects of CS on M1 low-frequency oscillatory activity during ketamine anesthesia; neural recordings during anesthesia are of substantially greater quality and can allow us to easily monitor spiking and LFP during stimulation. After anesthetic induction, we implanted epidural electrodes for stimulation and M1 microwire electrodes to measure neural activity (FIG. 4a). Baseline spiking/LFP activity was recorded for 15 minutes; this was followed by recordings during the application of a 1-5 minute long CS via the cranial screw adjacent to the implanted electrodes. Interestingly, we found that CS could effectively modulate ongoing LFO dynamics during ketamine anesthesia (FIG. 4b-c). More specifically, CS significantly increased LFP power in the lower frequencies, (FIG. 4b, example animal, p<0.05 across 10 animals comparing 1.5 to 4 Hz power pre-versus during stimulation). CS also increased neural SFC (FIG. 4c, p<0.01, n=51 neurons, from 1.5-4 Hz); SFC analyses controlled for any firing rate changes. These results indicate that CS can directly boost LFOs, i.e. a concomitant increase in both LFP power and the phase coupling of spiking activity.

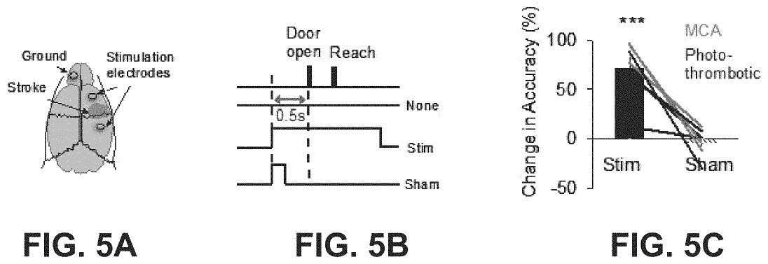

Having found that a low-strength electric field CS could modulate low-frequency oscillations, we next performed experiments to assess whether short pulse of CS (<5 seconds in duration) applied directly during the reaching behaviors could improve motor function after stroke. Importantly, we avoided the significantly longer-duration pulses (e.g. continuous for ≥10 minutes) that are known to induce long-lasting changes in excitability; we wanted to specifically assess whether transient on-demand stimulation could induce behavioral improvements. For these experiments, animals underwent either a photothrombotic (n=4) or distal-MCA (n=3) stroke induction and were implanted with cranial screws for stimulation both anterior and posterior to the injury site (FIG. 5a). Animals then underwent motor training for days to weeks until their level of performance plateaued; CS stimulation was then tested. Stimulation experiments occurred between 20 and 150 days after the stroke across animals, with no clear relationship between time after stroke and efficacy of stimulation. We compared the effects of stimulation with a “no-stimulation” and a “sham-stimulation” condition (FIG. 5b). Importantly, we clearly found that stimulation effects were truly “on-demand” and did not persist across blocks. Thus, for each daily session we could test all three conditions (i.e. blocks of trials of no stimulation, sham, stimulation). The order of these blocks was pseudo-randomized across days in every animal, and across sessions; we did not find that order of block affected results. We calculated the percent improvement in accuracy for each daily stimulation and sham condition relative to the no-stimulation condition for that day. Animals showed an improvement of 73±12% in accuracy following stimulation (one-sample t-test, t(6)=6, p<0.001) and a non-significant change of −4±5% in the sham stimulation group (one-sample t-test, t( )=−0.77, p>0.05). There was also a significant difference in the observed behavioral effects between the stim and sham conditions (paired-t test, t=4.9, p<0.01). We observed improvements in performance in both stroke models with no significant differences in the effects observed by stroke model type (F(1,5)=1.5, p<0.05). While the above experiments were conducted using cathodal stimulation, we found similar effects using anodal stimulation condition (anodal-stimulation showed an improvement of 60±12% (one-sample t-test, t(4)=4.9, p<0.01, n=5 animals). There was no difference between anodal and cathodal stimulation groups in the effect of stimulation on motor improvement (ANOVA, F(1,10)=1.35, p>0.05).

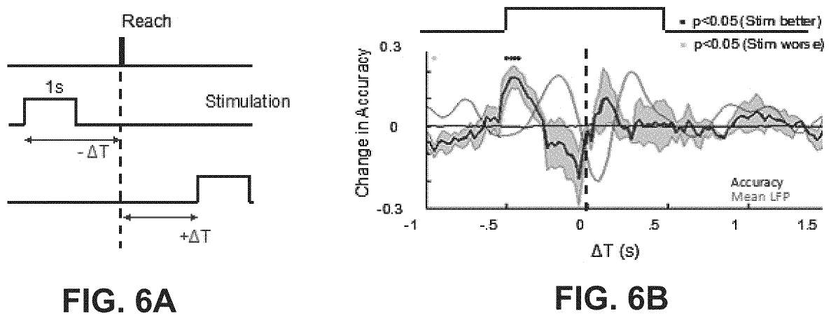

The results above used relatively long duration pulses relative to the duration of a typical reach-to-grasp movement (i.e. ˜700 ms). We also tested whether 1 second long stimulation pulses could allow us to more precisely determine the temporal relationship between electrical stimulation and the neural processes underlying reach control after stroke. For each reach trial, we randomly varied the precise timing of stimulation onset relative to when the door opened as a ‘Go’ cue (FIG. 6a). Importantly, the only parameter varied was the timing of the stimulation onset relative to this cue. Next, we calculated the ΔT between stimulation onset and the actual reach onset for each trial; this allowed us to account for variations in the reaction time. We then calculated the % accuracy for all trials at a particular ΔT by binning all trials in a window of ±100 ms around that time-point. Across 4 animals we observed a significant improvement in accuracy only when ΔT occurred between 500-400 ms from the reach (FIG. 6b, p<0.05, bootstrapped). Given that we used 1 second pulses, this indicated that stimulation pulses that started prior to reach onset and lasted through the duration of the reach were the most effective. Interestingly, this timeframe may be related to the expected period for task related LFOs. As a possible metric for comparison, we plotted the mean task evoked LFO (data from FIG. 3). Together, our data suggests that stimulation pulses that maximally overlapped with the neural dynamics prior to and during the reach were the most effective at improving function.

It is important to note that electrical stimulation can have differential effects related to the onset/offset of stimulation as well as during the “steady-state” or the DC field effect. This may explain the significant worsening that was observed for stimuli that started 0.975 seconds prior to reach onset (FIG. 6b green star; i.e. the offset was exactly at the time of reach onset). Perhaps also consistent with this interpretation is the finding that pulses that started immediately prior (i.e. <500 ms) to movement onset or during the movement did not result in consistent beneficial effects. Together with the results from FIG. 6, our results indicate that pulses that start at least 500 ms prior to reach onset and last through the reach are consistently able to improve reaching behaviors.

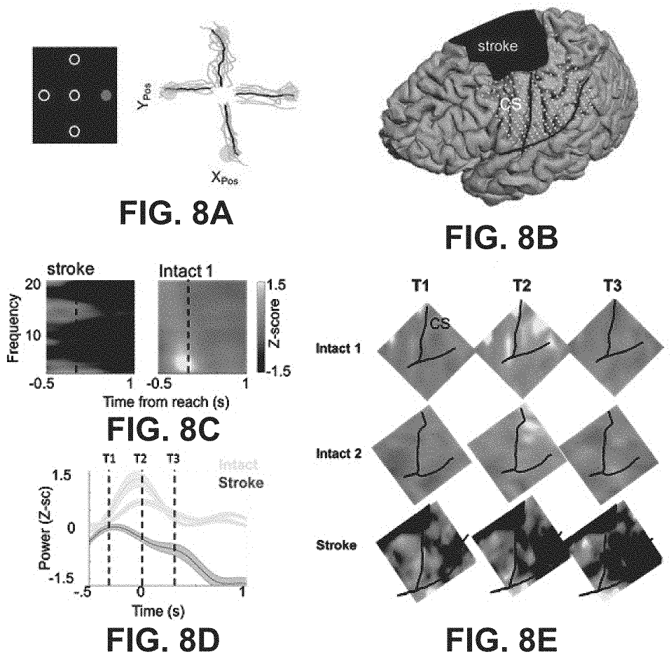

We next assessed whether our observed phenomena in rodent models could also apply to human stroke. In order to assess this, we reanalyzed human ECoG (ElectroCortiocoGraphy) data collected from human subjects undergoing invasive epilepsy monitoring. All subjects underwent invasive ECoG monitoring to identify seizure foci. Physiological data were recorded during a center-out reach task in which subjects were instructed to wait for a start cue and then reach as fast as possible to a target (FIG. 8a). Two of these patients had intact sensorimotor cortices (hereafter Intact Subjects or IS1/IS2) and the third had a cortical stroke particularly affecting the arm and hand motor areas (hereafter Stroke Subject or SS) (FIG. 8b). The stroke subject had persistent motor deficits involving arm and hand movements (Fugl-Meyer upper-limb score of 35). He also showed impairments in speed of execution. Reaction time from the “Go” cue to movement onset (i.e. rise in mean EMG activity) was slower for the affected versus unaffected arm (mean reaction time of 635±40 and 365±18 ms, respectively, P<0.001, unpaired t-test). Similarly, the reach time from movement onset to target acquisition was longer for the affected arm (mean reach time of 1266±58 ms vs. 856±26 ms, P<0.001, unpaired t-test).

With respect to the EcoG recordings for the two intact subjects, we found evidence for robust task-related LFOs centered around sensorimotor cortex (FIG. 6c). The time course and pattern of this activity appeared to closely resemble that observed in rodents. In the stroke subject, however, there was a striking loss of this sensorimotor reach-related low-frequency activity (FIG. 8c-e). The mean normalized LFO activity for sensorimotor electrodes (from −300 ms to +300 ms) was significantly positive for the two non-stroke subjects (Subject 1, normalized mean activity 0.55±0.2, n=18 SM electrodes, t(17)=7.2, p<0.001) and 0.93±0.25 in the second subject, (n=16 SM electrodes, t(15)=5.5, p<0.001), while the stroke subject showed no significant increase activity (−0.12±0.1, n=91 SM electrodes, t(90)=−1, p>0.05). There was a highly significant difference in task-related low frequency power between the stroke subject and the two healthy subjects, (F=9.8; p<0.001; post-hoc p<0.01, Bonferonni corrected, comparing stroke subject to each of the healthy subjects; post-hoc tests confirmed with bootstrapping implemented within SPSS). There was no difference between the two healthy subjects (p>0.05). Importantly, prior analyses of the data from the stroke subject demonstrated intact high-gamma activity in much of the brain, including sensorimotor cortex, despite motor deficits. High-gamma is widely thought to represent local spiking activity. These results suggest that low-frequency oscillatory activity is a common electrophysiological signature of healthy motor circuit function across both rats and humans, and in both species stroke appear to disrupt this task-related physiological marker even in brain areas demonstrating reach-related spiking activity (i.e task related spiking modulation in rats and high-gamma activity in the ECoG recordings).

### SINGLE TARGETING IN THE CORTEX

An emerging view of primary motor cortex (M1) sees it as an engine for movement governed by transient oscillatory dynamics present during both preparation and generation of movement. Movement-related, low-frequency quasi-oscillatory activity (LFO), at the level of both spiking and local field potentials (LFP), has also been observed in the intact non-human primate M1 and human motor regions during reaching tasks. Such quasi-oscillatory activity can be as brief as 1-2 cycles for rapid movements or longer during sustained movements, and appears to be closely correlated with sub-movement timing. They may also be related to the multiphasic muscle activations required for precise kinetics during actions. Thus, LFOs appear to represent an intrinsic property of motor circuits involved in the production of fast and accurate movements.

Here we hypothesized that monitoring and manipulating movement-related LFOs after stroke may offer new avenues to understand motor recovery. Prior research using invasive electrophysiological approaches has largely focused on measurements of nervous system function that occur at rest and/or away from motor tasks. For this reason, surprisingly little is known about how stroke and recovery affects task-related neural dynamics at the level of single neurons and mesoscopic circuit function. Non-invasive studies in human subjects have found that EEG movement-related potentials (e.g. slow-cortical potentials or SCPs) are affected by stroke. Furthermore, changes in SCP are correlated with motor impairments post-stroke. One limitation of EEG, however, is the uncertainty regarding specific anatomical generators and neural processes that contribute to the recorded potentials; moreover, SCPs include a variety of pre-movement and movement related phenomenon, further limiting their interpretation.

A generative model of cortical dynamics in both the healthy and recovering nervous system may guide the development of novel, closed-loop neuromodulatory approaches that dynamically target transient task-related processes. Despite our knowledge that neural networks are highly non-stationary, the vast majority of prior studies applying electrical or magnetic stimulation to the brain post-injury have applied it continuously, without explicitly targeting intrinsic neural dynamics and with a primary goal of generally increasing excitability and/or plasticity. However, recent work has suggested that therapeutic electrical stimulation can be used to target phasic oscillatory dynamics, an idea has been successfully implemented in Parkinson's disease and epilepsy. Implementing such an approach post-stroke requires detailed knowledge of normal and abnormal neural dynamics, and a better understanding of how to modulate them. Here we aimed to identify neurophysiological dynamics associated with skilled execution; assess whether these same dynamics are related to recovery; and finally, to evaluate whether temporally precise electrical neuromodulation of these dynamics can improve motor function post-stroke.

### RESULTS

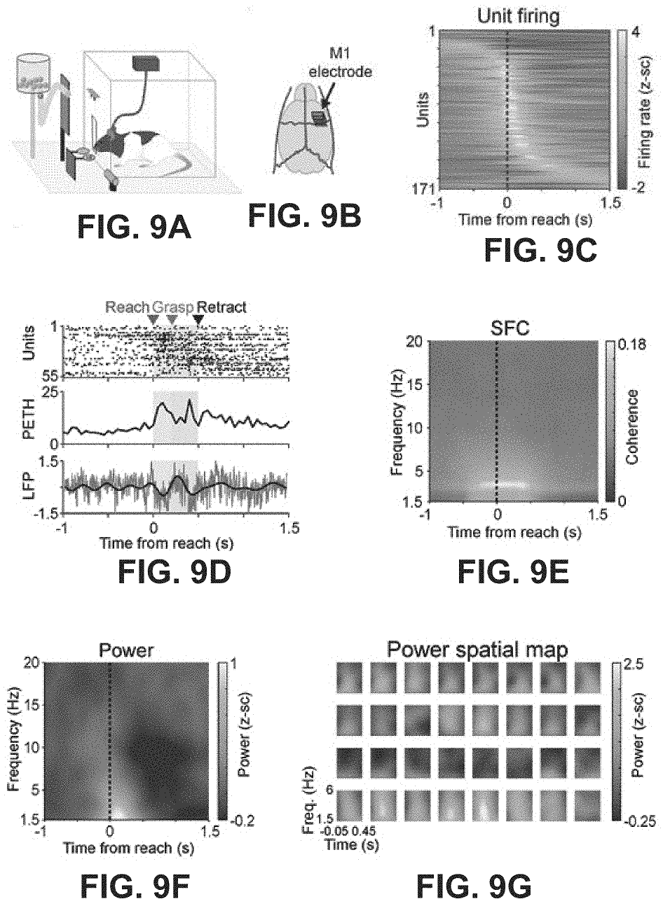

Long Evans male rats (n=4) were implanted with microwire arrays in M1 after learning a skilled forelimb reach task (FIG. 9a-b). Animals were trained over multiple days using an automated reach-box39. In addition to movement-related spiking activity in M1 in well-trained rats (FIG. 9c), we also observed quasi-oscillatory low-frequency activity at the level of both LFP and spiking activity (FIG. 9d, example trial). We found strong movement-related power predominately in lower LFP frequencies that began prior to reach onset; neurons showed coherent spiking with the LFP at these frequencies (FIG. 9e-f). We quantified these effects by calculating the mean 1.5-4 Hz LFP power and spike-field coherence or SFC (−0.25 to +0.75 s around reach onset) across channels/units from all animals. There was a significant increase in both power (mixed-effects model with 118 channels and 4 rats as random effect, t(117)=6.77, p=5.37e-10) and SFC (mixed-effects model with 170 units and 4 rats as random effect, t(170)=8.07, p=1.24e-13) during the reach as compared to the pre-reach “baseline”. Because power and SFC were computed for each trial and then averaged, these values are not related to the mean evoked “event related potential” or ERP, but rather to single-trial dynamics. Together, these findings indicate that rodent M1 also demonstrates similar task-related low-frequency quasi-oscillatory activity described in non-human-primates2-4,14. A dynamic increase in SFC associated with movement suggests one of two possibilities: single units and LFP could both be phase-locked to the motor action and thus simply appear phase-locked to each other; or, by contrast, there may be independent phase-locking between units and LFP. One approach of teasing this apart is to subtract out the average ERP, which represents the dominant “phase-locked” LFP activity across trials, and then recalculate power/SFC. By subtracting the ERP, we were left with “induced” oscillations (the non-phase-locked changes in power associated with movement); thus, the subsequent SFC measure indicates a more direct relationship between LFP phase and spiking that is less contaminated by phase-locked LFP activity to the reach. Using this approach, we again found a strong increase in task-evoked low-frequency SFC and power evoked by reaching.

One advantage of LFP recordings over spiking is stability over long-time periods. In contrast, spike recordings are easily affected by micro-motion, making it difficult to follow the same ensemble across days. Notably, we found remarkable stability in the measured task-related low frequency LFP power across trials and days. Finally, LFP measurements provide information about mesoscale organization of neural activity (FIG. 9g). Interestingly, we found that only a subset of channels demonstrated an increase in task-related low frequency power; there appeared to be spatial clustering of channels, suggesting that M1 activation is not uniform at the mesoscale level.

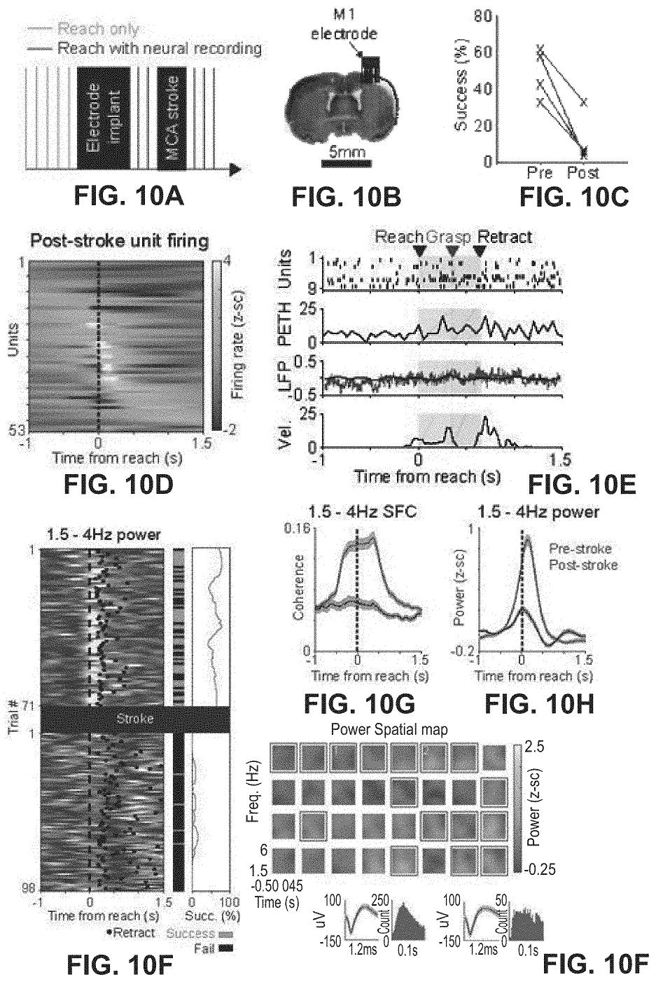

After collecting electrophysiological data in the healthy state (FIG. 9), we performed a distal MCA-occlusion stroke on these same animals (FIG. 10a). Induction of this type of stroke could be performed without perturbing implanted electrodes, thus allowing for a direct comparison of neural activity pre/post stroke in the same animals and cortical region. The distal-MCA model stroke resulted in a large area of damage within sensorimotor cortex (FIG. 10b). Animals were tested again after at least a 5-day rest post-stroke; neural activity was measured again once animals could attempt reaches and at least occasionally retrieve the pellet. The stroke resulted in impaired skilled motor function (FIG. 10c). Importantly, neural probes were positioned such that at least some electrodes remained in viable tissue (FIG. 10b); even post-stroke, single units remained on a subset of electrodes (FIG. 10d). There were fewer units post-stroke (average of 1.45 vs. 0.453 units/channel pre vs. post-stroke), but those that remained continued to demonstrate task-related increases in activity, though demonstrating significantly less modulation on average (FIG. 10d). Reach-related LFOs were perturbed (FIG. 10e-i). Low-frequency SFC was reduced after stroke (FIG. 10g, mixed effects model t(221)=7.45, p=2.07e-12); changes in firing rate could not explain the observed changes in SFC. To further probe the relationship between spiking activity and LFP using a method that is not confounded by potential changes in firing rate, we calculated the preferred phase of spiking. We found strong phase-locking to the trough of the low frequency LFP pre-stroke, and no preferred phase of spiking post-stroke. LFP power also reduced after stroke (FIG. 10h-i, mixed-effects model t(100)=6.01, p=3.06e-8. As task-related units were present, the loss of the reach-related LFP power was not simply a product of probes being in infarcted tissue (FIG. 10i). The decrease in LFP power was also not due to changes in movement speed; power was not correlated with movement duration. As before, subtracting the mean ERP to isolate “induced” activity did not significantly change results. Together, these analyses clearly demonstrated that stroke resulted in a striking loss of LFOs and phase-locked quasi-oscillatory spiking activity.

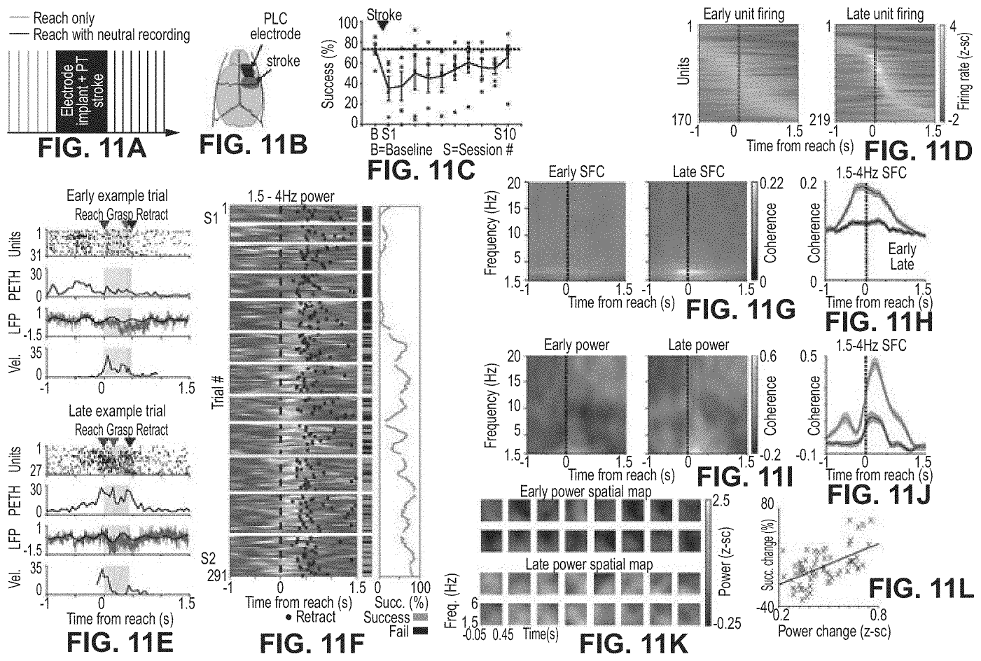

Having observed a clear decrease in LFOs in M1 after stroke, we next wondered if recovery of function might be associated with its restoration in peri-lesional cortex. Because of variability in the location of damage after distal MCA occlusion, we performed this next set of experiments using a focal photothrombotic stroke model to generate a relatively reproducible area of damage; hence allowing us to know a priori the location of the perilesional cortex and to target neural probes to the appropriate rostral location where rehabilitation-induced plasticity has been shown to occur. Immediately after stroke induction, a 16- or 32-channel microelectrode array was implanted anterior to the site of the injury (FIG. 11a-b). Animals were given 5 days to recover from the stroke and electrode implantation; they then underwent motor training on the same task to assess the relationship between recovery and task-related LFOs in perilesional cortex. Injury resulted in impaired motor performance (73.6%±12.21% vs. 35.1%±11.9%, 2-tailed paired t-test, t(5)=3.35, p=0.0204) which improved over the course of subsequent training (69.1%±9.01% last session; 2-tailed paired t-test comparing first vs last session, t(5)=3.03, p=0.0290; FIG. 11c.

With recovery of function, spiking activity in perilesional cortex became sharper, more task-related and more similar to that observed in the healthy M1 (FIG. 11d). There was a clear emergence of low-frequency task-related activity in both spiking and LFP in perilesional cortex (FIG. 11e-k). This increase in LFO can be observed in single trial examples (FIG. 11e) and across trials/sessions within the same animals (FIG. 11f). Statistically, there was a strong increase in 1.5-4 Hz SFC (FIG. 11g-h, mixed effects model t(387)=8.94, p=1.59e-17). Changes in SFC could not be explained by changes in firing rate. 1.5-4 Hz power also increased significantly (FIG. 11i-j, mixed effects model t(175)=3.11, p=0.00217. Moreover, subtracting the ERP did not change the results.

There was a significant positive relationship between the restoration of low-frequency power and improvements in accuracy on the task (FIG. 11f, example animal; FIG. 11I, all animals, Pearson's correlation r=0.576, p=1.18e-7). There was also a significant correlation between the restoration of SFC and recovery of function (r=0.554, p=4.60e-7) and between single unit modulation change and recovery (r=0.561, p=3.01e-7). A multi-variate linear regression model with all three variables significantly predicted motor improvements (r=0.737, p=1.28e-11). Each variable had significant partial correlation (r=0.428, p=2.21e-4 for power; r=0.339, p=0.00410 for SFC; r=0.398, p=6.29e-4 for unit modulation), suggesting that all variables could independently account for variance in recovery of function.

We next assessed whether our observed phenomena in rodent models were relevant in human stroke by reanalyzing invasive human ECoG (ElectroCortiocoGraphy) data collected from three human subjects undergoing invasive epilepsy monitoring to identify seizure foci. Physiological data were recorded during a center-out reach task in which subjects were instructed to wait for a start cue and then reach as fast as possible to a target (FIG. 8a). Two of these patients had intact sensorimotor cortices (hereafter Non-Stroke or NS1/NS2); the third patient, however, had experienced an ischemic cortical stroke four years prior to the monitoring (hereafter Stroke Subject or SS) (FIG. 8b). The stroke subject had persistent motor deficits involving arm and hand movements (Fugl-Meyer upper-limb score of 35). He also showed impairments in speed of execution. Reaction time from “Go” to movement onset (i.e. rise in mean EMG) was slower for the affected versus unaffected arm (reaction time of 635±40 and 423±72 ms, respectively, t(56)=−2.7, p=0.009, two-tailed two-sample t-test). Similarly, the reach time from movement onset to target acquisition was longer for the affected arm (reach time of 1266±58 ms vs. 914±51 ms, t(56)=−4.42, p=4.65e-5, two-tailed two-sample t-test).

For ECoG recordings from NS1/NS2, we found evidence for robust task-related LFOs centered around sensorimotor cortex (FIG. 8c). The time course and pattern of this activity (FIG. 8d) appeared to closely resemble that observed in rodents (FIG. 9f). In the SS, however, there was a striking loss of this sensorimotor reach-related low-frequency activity (FIG. 8c-e). The mean normalized 1.5-4 Hz LFP power for sensorimotor electrodes (from −300 ms to +300 ms) was significantly positive for the two non-stroke subjects: NS1, normalized mean activity 0.55±0.2 (n=18 SM electrodes, two-tailed one-sample t-test, t(17)=7.16, p=2e-6) and 0.93±0.25 in NS2, (n=16 SM electrodes, two-tailed one-sample t-test, t(15)=5.47, p=6.5e-5), while the stroke subject showed no significant increase in power (−0.12±0.12, n=91 SM electrodes, two-tailed one-sample t-test, t(90)=−1.03, p=0.304). There was a highly significant difference in task-related low frequency power between SS and NS1/NS2. We analyzed all channels from all subjects comparing healthy vs. stroke, including subject as a factor in the model to account for differences between the two healthy subjects. Using this approach, we found a highly significant overall effect (F(2,122)=9.80, p=1.13e-4, and more importantly, a highly significant effect of stroke (F(1,122)=18.76, p=3.1e-5). It is possible these results were observed because, while in healthy subjects, the LFO was dominant near the central sulcus, in stroke, due to cortical reorganization, the LFO could be observed in other regions of the brain. Indeed, prior analyses of the data from SS demonstrated intact high-gamma activity away from the central sulcus, that were correlated with muscle synergies, suggesting functional reorganization. To account for functional reorganization, we thus selected channels that showed increased activity in the high-gamma band between −300 to 300 ms prior to reach. This was performed blind to location, in an un-biased manner for all three subjects. Using this method of functional rather than anatomic selection, we found overall similar results. These results suggest that low-frequency quasi-oscillatory activity is a common electrophysiological signature of healthy motor circuit function across both rats and humans.

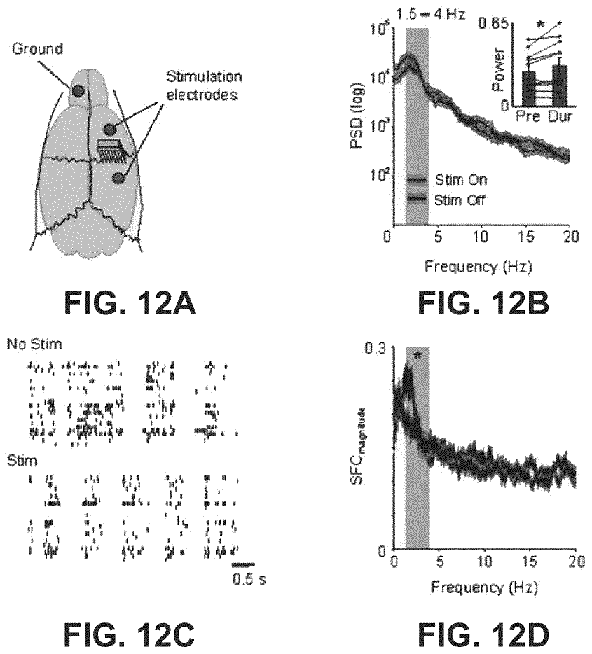

A key goal of this project was to assess whether we could modulate task-related oscillations and thereby develop a targeted neuromodulation approach post-stroke. Prior research has demonstrated that direct current stimulation (DCS) can modulate spiking activity and on-going, carbachol-induced gamma-oscillatory dynamics. It has also been recently reported that low-frequency oscillatory activity observed during ketamine anesthesia is similar to the brief, low-frequency spiking/LFP dynamics during natural reaching. To study the effects of DCS in vivo, we analyzed the effects of DCS on M1 low-frequency oscillatory activity during ketamine anesthesia (10 rats, 11 sessions). Neural recordings during anesthesia are of substantially greater quality; we can move electrodes to optimize location near neurons and greatly increase signal to noise, a requirement for monitoring spiking during stimulation. After anesthesia induction, we implanted epidural electrodes for stimulation and M1 microwire electrodes to measure neural activity (FIG. 12a). Baseline spiking/LFP activity was recorded for 15 minutes, followed by recordings during the application of a 1-5 minute long DCS (mean duration 2.909±0.607 mins, mean amplitude: 106.364±44.526 □A) via the epidural electrodes adjacent to the implanted recording electrodes. We found that DCS could effectively modulate ongoing LFO dynamics during ketamine anesthesia (FIG. 12b-d). Specifically, DCS significantly increased LFP power in the lower frequencies (FIG. 12b 1.5-4 Hz LFP power, baseline 0.266±0.047 and with DCS 0.314±0.062; two-tailed paired t-test t(10)=−2.49, p=0.032). DCS also generally increased phasic spiking (FIG. 12c) and significantly increased 1.5-4 Hz SFC (FIG. 12d, SFC without DCS: 0.278±0.016 and during DCS 0.316±0.022; one-tailed paired t-test t(49)=−1.73, p=0.0452). Moreover, 40% of neurons changed their firing rate significantly. More specifically, 30% increased and 10% decreased their firing rates over the baseline period. SFC analyses were performed after controlling for any firing rate changes. This was important as firing rate changed significantly for these neurons at a population level (n=50, two-tailed paired t-test, t(49)=−2.65, p=0.0109).

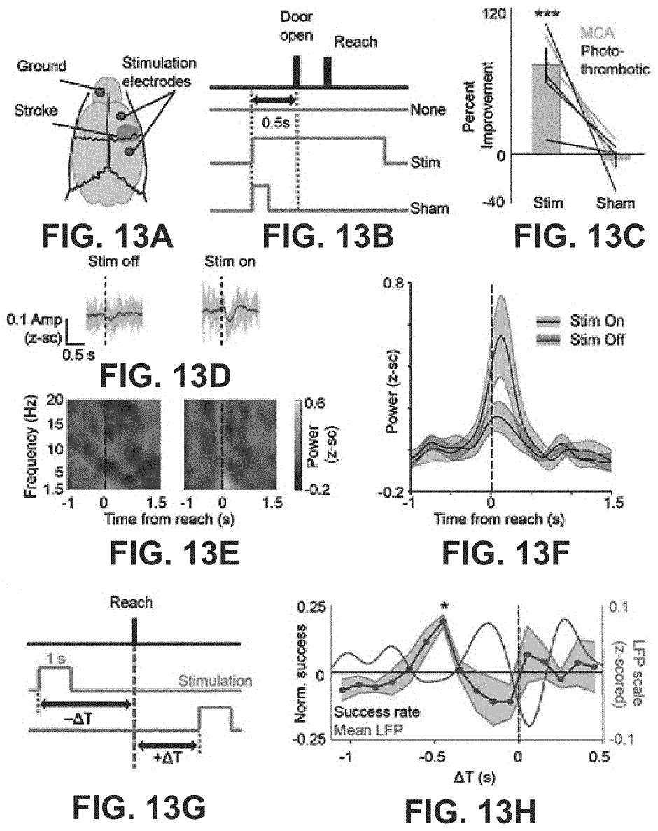

We next performed experiments to assess whether shorter pulses of DCS (<5 seconds in duration), applied directly during reaching behaviors could improve motor function after stroke. Importantly, we avoided the significantly longer duration stimulation (e.g. continuous stimulation for 5 minutes) that are known to induce long-lasting changes in excitability, as we wanted to specifically assess whether transient “on-demand” stimulation could induce behavioral improvements. For these experiments, animals underwent either a photothrombotic (n=4) or distal-MCA (n=3) stroke induction and were implanted with cranial screws for stimulation both anterior and posterior to the injury site (FIG. 13a). Animals then underwent motor training until their level of performance plateaued (see methods); DCS was then performed. Stimulation experiments occurred between 20-150 days after the stroke, with no clear relationship between time after stroke and efficacy of stimulation. We compared the effects of stimulation with a “no-stimulation” and a “sham-stimulation” condition (FIG. 13b). Using this paradigm, we found that stimulation effects were “on-demand” and did not persist across blocks, allowing us to test, daily, all three conditions (blocks of trials of no stimulation, sham-stimulation or stimulation). The order of these blocks was pseudo-randomized across days in every animal, and across sessions. We calculated the percentage improvement in accuracy for each daily stimulation and sham condition relative to the no-stimulation condition for that day, and then calculated the mean improvement across days for each animal to perform statistics. Animals showed an improvement of 73±12% in accuracy following stimulation compared with no stimulation (one-sample, two-tailed t-test, t(6)=6, p=9.6e-4) and a non-significant change of −4±5% in the sham stimulation group (one-sample, two-sided t-test, t(6)=−0.77, p=0.47, FIG. 13c). There was also a significant difference in the observed behavioral effects between the stim and sham conditions (two-tailed paired-t test, t(6)=4.91, p=0.003). Further analyses describing stroke-type and variation in effects across days as well as additional experiments using cathodal stimulation, are described in online methods.

We next assessed whether DCS could enhance task-related LFOs. We recorded neural signals from four post-stroke rats with persistent deficits, while they attempted the reach-to-grasp task over a total of 24 sessions (total of 1031 trials, 532 reach trials with ‘Stim On’ and 499 trials without DCS). Simultaneous recording of neural signals during brief epochs of stimulation is particularly challenging as the stimulation onset/offset triggers large distortions in both LFP and spiking. We thus had to substantially alter the stimulation parameters. We used significantly lower current amplitudes (81.654±12.414 μA vs 321.4±12.2 μA in behavioral experiments above), longer duration pulses (DCS pulses were typically 15 seconds long) and more distant stimulation sites to accommodate recording probe (see methods). The average z-scored 1.5-4 Hz LFP power was higher during DCS trials (0.201±0.076) compared to no stimulation trials (0.059±0.038, t(1029)=7.425, p=2.361e-13, mixed effects model, FIG. 13d-f). We observed a trend towards increased accuracy with DCS in this set of animals 21.069±14.963% increase (one-tailed paired t-test, t(3)=−1.830, p=0.082). The reduced efficacy was likely the result of the lower current amplitude used. Consistent with this notion is the data from our early pilot experiments (see Methods) and in the behavior-only animals (FIG. 13c) where stimulation currents of >150 μA per screw were required to observe consistent behavioral improvements.

Lastly, we designed a separate set of stimulation experiments using one second long pulses in a new group of animals to replicate the prior effect and more precisely determine the temporal relationship between electrical stimulation and the neural processes underlying reach control after stroke. More specifically, we pseudo-randomly varied the timing of stimulation onset (in blocks of 25 trials) relative to the trial onset (i.e., door opened to allow reach) (FIG. 13g). Importantly, the only parameter varied was the timing of the stimulation onset relative to this cue; stimulation was delivered on all trials. Next, we calculated the ΔT between stimulation onset and the actual reach onset for each trial, thereby allowing us to precisely assess the relationship between the timing of stimulation and change in motor function. We then calculated the % accuracy for all trials at a particular ΔT by binning all trials in a window of ±50 ms around that time-point (100 ms bins). We observed a significant improvement in accuracy only when ΔT occurred between 500-400 ms from the reach (FIG. 13h, two-tailed, one-sample t-test, t(3)=9.035, p=0.0458, Bonferroni-Holm correction for 16 time points). It is important to note that, with 1 second pulses, stimulation around this time point is likely to maximally overlap with the expected LFO (visualized on the plot, though the mean LFP trace was taken from different animals). Given the brief duration of stimulation pulses, 1 second long stimulation pulses at other times were likely to begin or end during the LFO; and, interestingly, did not appear to be beneficial. Together, our data demonstrates that DCS improved motor function in a temporally restricted manner and could enhance the LFO after stroke, suggesting a novel mechanism by which neuromodulation can work to improve motor function post-stroke.

### DISCUSSION

Our results identified low-frequency quasi-oscillatory activity as an important neurophysiological marker of skilled motor control. We found evidence of such activity at the level of neural spiking and LFP during the performance of a dexterous task in rats, and in ECoG signals in human subjects without stroke. In both rodents and humans, cortical stroke appeared to significantly disrupt low-frequency activity and its reemergence strongly tracked recovery of motor performance in rats. We also found that pulses of electrical stimulation enhanced entrainment of spiking, increased LFOs, and also improved motor performance in animals with persistent deficits. Consistent with this model, electrical stimulation was primarily effective when it started prior to and lasted through the reach, suggesting that applied electrical fields directly modulated neural dynamics linked to task execution.

There is growing literature demonstrating that quasi-oscillatory low-frequency activity can capture reach dynamics; our results provide evidence that this activity is relevant during recovery as well. Are these events truly “oscillatory”, given their relatively brief nature? In this study, we used an established analytic framework for time-frequency decomposition of motor evoked activity to assess the spectral content of evoked activity. Using these methods, we were able to (1) quantify the relationship between spiking and LFP (i.e. SFC), (2) develop a model for how DC stimulation effects neural circuits, and (3) link our findings with human ECoG recordings. All of this suggests that LFOs provide a useful framework for characterizing important cortical dynamics during recovery. A final point in favor of this framework is that we found significant partial correlations between behavioral improvements separately for both SFC and low frequency LFP power; this suggests that specific aspects of the oscillatory dynamics (spiking and LFP) provide independent explanatory power about motor recovery. This does raise a concern regarding the correct interpretation of the SFC. Specifically, task-evoked SFC could arise simply because both LFP and spiking are phase-locked to behavior, even if they are not directly related to each other. To address this, we subtracted the average ERP, which represents the phase-locked component of the LFP; we still observed task-related increase in power and SFC, suggesting the two signals are related to each other, and not simply similarly phase-locked to behavior. Together, our results indicate that restoration of oscillatory dynamics observed both in spiking and LFP data, is important for motor recovery.

What is the possible relationship between LFOs, skilled behaviors, and motor recovery? Low-frequency oscillations can be used to decode reach-related activity and predict spiking phase across multiple behavioral states. Such activity is also correlated with multiphasic muscle activations and movement timing. Recent work also suggests that oscillatory dynamics reflect an underlying dynamical system. This prior work argues that LFOs represent an intrinsic property of motor circuits associated with precise temporal control of movements. Our findings extend this body of work by linking restoration of LFO dynamics in perilesional cortex to motor recovery. Our results directly implicate LFOs in the re-instantiation of cortical control of complex limb dynamics during reaching. In our human stroke subject, persistent loss of cortical LFOs may suggest a mechanism for why reaching behaviors continued to be impaired. Of course, as we were only able to get data from one stroke patient, the generalizability of these findings remains unknown. The results need confirmation in a larger cohort. Nonetheless, given the concordance with our extensive rodent-based investigations, it is reasonable to propose that recovery of LFOs may represent a marker of restored circuit dynamics after stroke important for skilled reaching.

The exact origin of LFOs and underlying generators remains unknown. While our finding that a focal cortical stroke can perturb LFOs might indicate a local source, it is also increasingly clear that local perturbations can affect large-scale networks. Indeed, reach-related LFOs may involve striatal or thalamocortical activity; with impairments and recovery after stroke a function of network plasticity rather than local effects restricted to M1. It is possible that these LFOs are related to slow-cortical potentials associated with actions measured using EEG. However, because those potentials may involve multiple cortical/subcortical networks, it is difficult to directly compare to our observed phenomenon. Further work specifically probing interactions between perilesional cortex and the broader motor network can clarify what drives our observed electrophysiological changes during recovery.

We found that pulses of DC stimulation (i.e. FIG. 13) could improve motor function when timed to start prior to and last through the reach period. How might electrical stimulation improve motor function after stroke; and how does this differ from prior neuromodulation methods in stroke? In many prior animal and human studies (best exemplified in the EVEREST trial), sub-threshold high-frequency epidural stimulation over perilesional cortex was used to generally enhance cortical plasticity. Stimulation was delivered for an extended period of time in an ‘open-loop’ manner, i.e. not-timed with behavior, and the primary outcome measures were long-term changes in map plasticity (in animals) and long-lasting changes in motor function (in both animals/humans). Such stimulation protocols are thought to induce lasting changes in excitability that likely requires BDNF. Another form of stimulation used a closed-loop paradigm in which stimulation in one region was linked with firing activity in different region, but again the primary goal was to induce long-term changes in network-plasticity. In contrast to these prior efforts, our study was designed to test whether electrical stimulation could specifically modulate the brief, movement-locked neural activity identified here and thereby improve motor function, i.e. apart from any long-term changes in cortical excitability or plasticity. Indeed, we show that brief, DC pulses can modulate movement-locked low-frequency activity and can improve motor function post-stroke. Our study, therefore, provides a basis for designing a rationale, on-demand and neurally-targeted stimulation paradigm for improving motor function. Moreover, our method of delivering stimulation (i.e. via cranial-screws) is potentially translatable as a class of invasive medical device. Such a device could address growing concerns that non-invasive stimulation may not reliably modulate cortex.

Stroke is one of the primary causes of long-term motor disability. Most current therapies, including task-specific rehabilitation training, are designed to enhance endogenous neural plasticity. Here we have identified a neurophysiological target and tested a dynamic neuromodulation approach for improving motor function post-stroke. Moreover, because LFOs can be recorded in human subjects both non-invasively (i.e. task-evoked delta/theta power using EEG) and invasively (i.e. using ECoG) there is a potential path to translate our results to stroke patients. These results may provide the basis for a new generation of “smart” stimulation devices that can precisely target neuromodulation to improve motor function after stroke.

### METHODS

### Animal Care and Surgery

All procedures were in accordance with protocols approved by the Institutional Animal Care and Use Committee at the San Francisco Veterans Affairs Medical Center. Adult male Long Evans rats (n=34, 250-400 g, Charles River Laboratories) were housed in a 12 h:12 h light: dark cycle. All surgical procedures were performed using sterile technique under 2-4% isoflurane or a ketamine/xylazine cocktail. Surgery involved cleaning and exposure of the skull, preparation of the skull surface (using cyanoacrylate), and then implantation of skull screws for referencing, stimulation and overall head-stage stability. Reference screws were implanted posterior to lambda, ipsilateral to the neural recordings. The ground screw was placed in the skull contralateral to the neural recordings and either placed posterior to lambda or over the nasal bone. For experiments involved physiological recordings, craniotomy and durectomy were performed, followed by implantation of neural probes. The postoperative recovery regimen included administration of buprenorphine at 0.02 mg/kg b.w for 2 days, and meloxicam at 0.2 mg/kg b.w. dexamethasone at 0.5 mg/kg b.w and trimethoprim sulfadiazine at 15 mg/kg b.w for 5 days. All animals were allowed to recover for one week prior to further behavioral training.

### Behavior

Animals were acclimated and then trained to plateau level of performance in a reach to grasp single pellet task before neural probe implantation. Probe implantation was performed contralateral to the preferred hand. Animals were allowed to rest for 5 days before the start of experimental/recording sessions. During behavioral assessments, we monitored the animals and ensured that body weights did not drop below 90% of the initial weight.

We used an automated reach-box, controlled by custom MATLAB scripts and an Arduino micro-controller. This setup required minimal user intervention, as described previously. Each trial consisted of a pellet dispensed on the pellet tray; followed by an alerting beep indicating that the trial was beginning and then the door opening. Animals then had to reach their arm out, grasp and retrieve the pellet. A real-time “pellet-detector” using an IR detector centered over the pellet was used to determine when the pellet was moved, indicating the trial was over, and the door was closed. All trials were captured by video, which was synced with electrophysiology data using Arduino digital output. The video frame rate was 30 Hz for the animals in the photothrombotic stroke electrophysiology experiments (n=6), and 75 Hz for those in the MCA stroke electrophysiology experiments (n=4) and stimulation experiments (n=14). Physiological data presented in this paper were generally time-locked to the onset of the reach movement. Onset of reach was determined manually from recorded video, and defined as the start of paw advancement towards the slot.

### In Vivo Electrophysiology

We recorded extracellular neural activity using tungsten microwire electrode arrays (Tucker-Davis Technologies). We used either 16- or 32-channel arrays (33 μm polyamide-coated tungsten microwire arrays). Arrays were lowered down to a depth of ˜1200-1500 μm. In healthy animals, neural probes were centered over the forelimb area of M117, at 3 mm lateral and 0.5 mm anterior from bregma. In photothrombotic stroke animals, the neural probe was placed immediately anterior to the stroke site, typically centered around 3-4 mm anterior and 2.5-3 mm lateral to bregma.

Units and LFP activity were recorded using a 128-channel TDT-RZ2 system (Tucker-Davies Technologies). Spike data were sampled at 24414 Hz and LFP data at 1017.3 Hz. ZIF-clip-based analog headstages with a unity gain and high impedance (˜1 GΩ) were used. Threshold for spiking activity was set on-line using a standard deviation of 4.5 (calculated over a 1 minute period using the TDT-RZ2 system), and waveforms and timestamps were stored for any event that crossed that threshold. Sorting was performed using Plexon OfflineSorter v4.3.0, using a PCA-based method followed by manual inspection and sorting. We included both clearly identified single-units and multi-unit activity for this analysis (results were pooled as there were not clear differences in single and multi-unit responses). A total of 171 single and multi-units were recorded from healthy animals, 53 from those same animals post MCA stroke, 170-219 from animals after photothrombotic stroke, and 50 units in the ketamine experiment (only single units with SNR>5.5 were used in this DC stimulation experiment in order to minimize stimulated-related contamination of neural signals). Behavior-related timestamps (i.e., trial onset, trial completion) were sent to the RZ2 analog input channel using an Arduino digital board and synchronized to neural data.

### MCA Stroke

For this procedure, adult rats were placed in the supine position, and a ventral cervical midline skin incision was made under the surgical microscope. Both the common carotid arteries (CCAs) were carefully isolated from the adjacent vagus nerve. The animal was then placed in the lateral position, and an incision was made over the temporalis muscle, which was then retracted. The main trunk of the left middle cerebral artery (MCA) was exposed and occluded with an AVM micro clip (Codman & Shurtleff, Inc., MA) and the CCAs was occluded using micro clamps, both for 60 minutes. After ischemia, micro clip and micro clamps were removed to restore blood flow after which the wound was sutured. This procedure has been previously shown to result in long-term loss of cortical tissue, and long-term impairments in motor cortical function 61.

### Photothrombotic Stroke and Electrophysiology

After craniotomy, rose-bengal dye was injected into the femoral vein using an intravenous catheter. Next, the surface of the brain was illuminated with white light (KL-1500 LCD, Schott) using a fiber optic cable for 20 minutes. We used a 4 mm aperture for stroke induction (centered in the M1 area based on stereotactic coordinates) and covered the remaining cortical area with a custom aluminum foil mask to prevent light penetration. After induction, a probe was implanted in the perilesional cortex (PLC) immediately anterior to the stroke site. The craniotomy/implanted electrodes were covered with a layer of silicone (Quiksil), followed by dental cement.

### Direct Current Stimulation (DCS)

### Anesthesia (Ketamine) Experiment

Animals (n=10) were initially anesthetized using a ketamine/xylazine cocktail (85 mg/kg ketamine, and 10 mg/kg xylazine), with supplemental ketamine given ˜every 40-60 minutes as needed to maintain a stable anesthetic level, and also to maintain anesthesia at stage III characterized by predominantly slow oscillations62; 0.05 mg/kg atropine was also given separately to help decrease secretions and counteract cardiac and respiratory depression. After anesthesia and craniotomy was performed, epidural stimulation electrodes were implanted (using skull-screws embedded in the skull), in the configuration noted in FIG. 12. The ground screw for this and all other stimulation experiments was implanted over the contralateral nasal bone, suggesting current flow would likely go through cortex and associated pathways in an anterior-medial direction from the site of stimulation. These screws were connected to a Multi-Channel Systems Stimulus Generator (MCS STG4000 series) to deliver direct-current stimulation. In 3 animals, ˜2 mm tungsten wire was placed on epidural surface in the craniotomy well instead of using skull screws to deliver the electrical stimulation. 32-ch multi-electrode arrays were implanted into Layer 5 of motor cortex (1200-1500 μm deep). Single-unit and LFP activity was recorded for 1 hour to ensure stability of recordings and minimize drift during stimulation experiment. Then, we recorded a base-line period of neural activity (˜15 minutes), followed by neural activity during direct-current stimulation (typically using 10-100 μA currents, applied for 1-5 minutes).

### In Vivo DCS Experiments

### Fixed Stimulation-Behavioral Experiments

After a stroke was induced (photothrombotic n=4 and distal-MCA n=3), two stainless steel skull-screws were implanted 1 mm anterior and posterior to the stroke site; we ensured that the electrodes were as close as possible to the stroke site and that they were located near the midline of the stroke area. Ground screw was implanted over contra-lesional nasal bone. Following a one-week recovery period animals were tested several times each week and those showing no persistent motor deficit (n=3) were excluded from further testing. Animals were tested until their behavior was at a plateau, with reach accuracies at least >15%. Direct-current stimulation, applied using an IZ2 stimulus isolator (TDT), was administered on both variable and fixed schedules. Stimulation was delivered on 2 screws in each animal, with a maximum stimulation amplitude of 200 μA/screw. Pilot studies in the first two animals suggested that accuracy on the skilled forelimb reach task was improved with >150 μA of current/screw; based on this pilot data, we provided at least 150 μA of current/screw in all animals undergoing behavioral testing. Stimulation current was increased up to the point of tolerability by the subject; with a max amplitude of 200 μA/screw. Tolerability was defined as animals not making any observable behavioral response to the onset/offset of stimulation pulse. We tested both cathodal and anodal polarities of stimulation, as described in results and below.

The current densities used in our study appear to be less that what has been used in previous studies. For example, a 2016 study used epidural electrodes for language mapping. The authors report using 5-15 mA of current delivered through 2.3 mm electrodes (area of 4.15 mm2); this results in a current density of 2.4 mA/mm2. Similarly, the current densities used for epidural stimulation in the Everest Trial were also comparable. The study reported using currents up to 13 mA using four electrodes with 3 mm diameter. Thus, each electrode could have a density of 0.46 mA/mm2. There are also multiple non-human primate studies using epidural stimulation. We estimate the following densities for the two example studies: 0.92 mA/mm2 64 & 1.41 mA/mm2. In comparison, we used 1 mm diameters screws. We typically used between 150-200 μA/skull screw when delivering stimulation. Our estimated current density was 0.25 mA/mm2. Thus, to the best of our knowledge, our current densities are comparable to those used in invasive human and non-human primate studies. Fixed stimulation (n=7, i.e. FIG. 13a-c data) began 500 ms prior to the door opening (i.e. signal of trial starting), and lasted up to 5 s total (encompassing the entire reach period, with stimulation turned off after the trial ended). 30-trial blocks of stimulation “on,” “off” and “sham,” (a 200 ms pulse that ended prior to the door opening, to mimic the sensory or possible alerting effects of the stimulation onset) were counterbalanced and interleaved across days. Effects of stimulation and sham were made based on percent improvements compared to temporally adjacent no-stimulation blocks. We made a decision to randomize at the level of blocks (i.e., blocks of 30 trials; 25 trials in DC Stim with physiology experiments) rather than at the level of trials because of pilot data (in 2 animals) that there were more robust behavioral effects when randomized in this manner.

Because we performed stim/sham stim sessions across days, we also calculated the standard deviation in the percentage improvement for each animal across days to see if this differed between conditions. We did not find a significant difference between the two conditions (t(6)=1.37, p=0.21). We did observe improvements in performance in both stroke models with no significant differences by stroke model type (t(5)=1.24, p=0.271). While the above experiments were all conducted using cathodal stimulation, we found similar effects using anodal stimulation condition (anodal-stimulation showed an improvement of 60±12% (one-sample t-test, t(4)=4.95, p=0.008, n=5 animals, which included experiments performed in two of the animals used above for cathodal stimulation and 3 additional animals, all in a photothrombotic stroke model). There was no difference between anodal and cathodal stimulation on motor improvement (ANOVA, t(10)=0.736, p=0.479).

### Joint Stimulation-Physiology Experiments

In studies combining electrophysiology and DC stimulation (FIG. 13d-e, n=4), we found that high stimulation currents resulted in artifacts that were difficult to remove. For this reason, we utilized smaller currents (81.654±12.414 μA mean current amplitudes in these experiments vs 321.4±12.2 μA in behavioral experiments above), with the primary goal of understanding whether DC stim could affect the LFO in any way. DC stimulation started 9 seconds before the door opened for the reach to start, and lasted 7 seconds after the door opened in these experiments, to minimize stim-related artifact in LFP recordings of interest (n=4 rats, i.e. FIG. 13d-e data). Photothrombotic stroke was used in the joint stimulation and physiological recording experiments (n=4). Furthermore, since the aim was to see if LFO was boosted with DCS, in these experiments, we started these experiments immediately after stroke (after a 14 day recovery period). For all fixed stimulation DCS experiments, the stimulation screws were placed anterior/posterior to the lesion/electrodes, and the “ground screw” was placed on the contra-lateral hemisphere on the nasal bone. For the joint stimulation and physiology experiments, the stimulation screws were placed somewhat diagonally and at further distance from stroke to accommodate recording array. Thus, the fixed stimulation versus joint stimulation and recording were optimized for behavioral effects versus physiologic recordings/effects respectively.

### Variable Stimulation Experiment

Variable timing stimulation (FIG. 13 f-g, n=4) began at six time-points with respect to door-open (−1 s, −0.5 s, 0 s, 0.5 s, 1 s, 1.5 s) and lasted 1 second to ensure a spread of temporal relationships between stimulation start and reach onset (ΔT). Stimulation was delivered in blocks of 25 trials with stimulation start time consistent within-block. Animals underwent 12 random-ordered blocks each day with each time-point tested in a total of 50 trials in two non-consecutive blocks. For each trial in each animal we calculated the exact time between stimulation and reach onset (ΔT) for analysis. Data was pooled in each animal from both anodal and cathodal stimulation experiments; there was no evidence that one form of stimulation worked consistently or significantly more than the other, consistent with data from the longer-duration stimulation experiments described above. Because there is some variability between the trial start (i.e. door opening), and the actual reach onset, the exact ΔT varied quite a bit from trial to trial even in the same stim block, thus helping to increase the randomization of this experiment.

### Immunohistochemistry

Rats were anesthetized and transcardially perfused with 0.9% sodium chloride, followed by 4% formaldehyde. The harvested brains were post-fixed for 24 hours and immersed in 20% sucrose for 2 days. Coronal cryostat sections (40 μm thickness) were incubated with blocking buffer (10% Donkey serum and 0.1% Triton X-100 in 0.1 M PB) for 1 hr, and then incubated with mouse anti-NeuN (1:1000; Millipore, Billerica, Mass.) for overnight. After washing, the sections were incubated with biotinylated anti-mouse IgG secondary antibody (1:300; Vector Lab, Burlingame, Calif.) for 2 hrs. Sections were incubated with avidin-biotin peroxidase complex reagents using a Vector ABC kit (Vector Labs). The horseradish peroxidase reaction was detected with diaminobenzidine and H2O2. The sections were washed in PB, and then mounted with permount solution (Fisher scientific) on superfrosted coated slides (Fisher Scientific, Pittsburgh, Pa.). The images of whole section were taken by HP scanner, and the microscope image was taken by Zeiss microscope (Zeiss, Thornwood, N.Y.).

### Human ECoG Experiments

As previously described, these studies were conducted using a protocol approved by the UCSF CHR; all studies were conducted after obtaining informed consent from subjects. Data were collected from two subjects without stroke and one subject with documented cortical stroke. All subjects had epilepsy, and had chronic ECoG grids implanted for pre-surgical monitoring/localization of seizure. All subjects performed a center-out reaching task, in which trials began with the appearance of a target at the center of the reach field, followed, after a variable delay, with a cue indicating subjects should perform a reach to one of 4 targets.

### Data Analysis

### LFP/ECOG and Single-Unit Analyses

Analyses were conducted using a combination of custom-written routines in MATLAB 2015a/2017a (Math Works), along with functions/routines from the EEGLAB toolbox (http://sccn.ucsd.edu/eeglab/) and the Chronux toolbox (http://chronux.org/). Pre-processing steps for LFP/ECOG analysis included: artifact rejection (removing broken channels and noisy trials); z-scoring; and common-mode referencing using the median signal (at every time-point, the median signal across the remaining electrodes, was calculated; and this median signal was subtracted from every channel to decrease common noise and minimize volume conduction). We used median referencing rather than mean referencing to minimize the effect of channels with high noise/impedance that were not discarded). For the joint stimulation and physiology experiments, we witnessed crosstalk between channels in two animals, and thus non-median subtracted LFP was analyzed. Filtering of data at specified frequency bands was performed using the EEGLAB function eegfilt( ) Calculation of power was performed with wavelets using the EEGLAB function newtimef( ) All time-frequency decompositions were performed on data on a trial by trial basis to capture the “total power” (that is, both the phase-locked, i.e., “evoked” and non-phase-locked, or “induced”) power. To isolate and also study only the “induced” oscillatory activity, we performed a similar analysis after subtracting the mean evoked potential from the single trial data. By subtracting this out, we removed on each trial the predominant phase-locked activity in the LFP, and what remained was the “induced” activity in which power is increased in a non-phase-locked way. Channels used for ECoG analysis were chosen by locating, for each subject, the central sulcus and selecting anatomically adjacent electrodes both anterior and posterior to the central sulcus. We performed the analysis using electrodes as far ventral as the Sylvian fissure for this paper; however, we also performed an analysis in which we subsampled only the dorsal half of these electrodes from each subject presumably closer to the hand knob, and found similar results.

Statistical quantification of how stroke/recovery affected power and spike-field-coherence in rodents was calculated by taking the mean power/SFC from −0.25 s to 0.75 s around reach onset. Only trials where the rat managed to at least touched and knocked off the pellet were included in the analysis. In FIG. 9, the baseline period is −3 to −2 s relative to reach onset. In FIG. 10, quantifications were made between all valid trials (at least 50) in the recording block before and after stroke. In FIG. 11, comparisons were made across the first 50 and last 50 trials or the first and last recording block, for power and SFC respectively, for each animal. In humans, we used data from −0.3 s to +0.3 s from reach onset across all trials performed in each subject. Calculation of spike-field coherence values was performed using the Chronux function cohgramcpt. For awake task-related experiments, SFC calculations were performed using 1 s windows moving by 0.025 s. For the anesthetized DCS experiments, multitaper and window parameters used for sleep-epoch analyses were utilized.

Sorted spikes were binned at 20 ms unless otherwise stated. After spikes were time-locked to behavioral markers, the peri-event time histogram (PETH) was estimated by Bayesian Adaptive Regression Splines (BARS). Unit modulation was calculated as (max−min)/(max+min) firing rate from −4 to 2.5 s around reach, after spline-fitting. Gaussian process factor analysis (GPFA) was done using DataHigh69, with spikes from −1 s to +1.5 s around grasp onset.

Spike-phase histograms in FIGS. 11 and 8 were calculated by first taking the Hilbert transform of the LFP filtered from 1.5-4 Hz, and then finding the phases of the LFP at which spikes (between −0.25 and +0.75 seconds from reach onset) occurred. For every spike-LFP pair (all spikes and LFP channels from each animal, across all 4 animals), we calculated the Rayleigh's z-statistic for circular non-uniformity, and then obtained the percentage of significant pairs (p<0.05).

### Statistical Analysis

Parametric statistics were generally used in this study (ANOVA, t-tests, Pearson's correlation and linear regression, unless otherwise stated), implemented within either MATLAB or SPSS. Linear mixed effects model (implemented using MATLAB fitlme) was used to compare the differences in unit modulation, SFC and LFP power in FIGS. 9-10 and the LFP power for stimulation on and off trials in FIG. 13. This model accounts for the fact that units, channels or trials from the same animal are more correlated than those from different animals, and is more stringent than computing statistical significance over all units/channels/trials. We fitted random intercepts for each rat, and reported the p-values for the regression coefficients associated with pre/post stroke, early/late recovery or stimulation on/off.

In FIG. 8, we used anatomically defined sensorimotor electrodes (electrodes that laid on either side of the central sulcus), and performed an ANOVA between conditions (stroke vs. non-stroke), with subject included as an additional factor. In FIG. 12, we analyzed data from only one channel in each animal (non-referenced), and calculated parametric statistics across animals (5b) or units (5d). In FIG. 13, we performed parametric statistics across animals. In FIG. 13f-g, to calculate significance, we performed two-tailed, one-sample t-tests at each time point displayed followed by Bonferroni-Holm correction for family-wise error. To confirm the effect, using a permutation test, we performed the following analysis. For each trial in each animal we calculated the time between stimulation and reach onset (ΔT) and the accuracy (success/fail) of that trial. We then randomized the accuracies relative to the (ΔT) 1000 times for each animal, maintaining in each animal the overall distribution of times (i.e. ΔT and accuracy. Then we computed for each animal the percentage accuracy at any particular ΔT (around a window of ±50 ms); and also the 1000 surrogate (i.e., randomized) accuracy at these time points. Across animals, we then calculated the mean accuracy, and compared this to the distribution of mean accuracies across the 4 animals generated from the randomized surrogates. Significance was assigned according to 2-tailed probabilities, such that at any point in time, accuracy > or <the 97.5th percentile in either direction at that particular ΔT was assigned a significance of <0.05. The significance values derived from this approach are more conservative than p-values derived from a more standard one-sample t-test at each time point, and likewise confirmed significance of the time-point in question (−450 ms prior to reach onset).

### MULTIPLE TARGETS IN THE MOTOR CORTEX AND STRIATUM

The act of reaching and grasping an object requires the precise coordination of both “gross” movements of the arm and “fine” movements of the fingers. Each of these distinct body parts, or “effectors”, plays a different role in the action and has distinct complexities in its control. For example, there are distinct degrees-of-freedom in movements of the arm and hand. How, then, does the nervous system coordinate such effectors to produce a unified skilled action? It has been suggested that such multi-effector coordination is achieved by globally optimizing movements with respect to biologically relevant task goals. For example, in reaching and grasping, both fine and gross movements may be jointly optimized to achieve task success while minimizing parameters such as effort. Surprising little, however, is known about the emerging neural basis of such coordination during skill learning.

While many tasks have been used to study the neural basis of skill learning (e.g. reaching and grasping, lever pressing, accelerating rotarod), learning is typically measured by task parameters rather than changes in the actual movements involved. For example, while rodent reach-to-grasp skill learning requires the coordination of both fine and gross movements, learning is commonly assessed using overall success rate rather than detailed movement analysis. Thus, a key goal of this study was to establish how changes in parameters such as success rate are achieved through changes in the coordination of the underlying movements involved and, further, to determine the neural basis for such coordination.

One possibility is that the emerging neural basis of multi-effector coordination reflects theories positing the global optimization of movements, i.e., a global neural controller emerges with training to control movements across effectors. In this case, during reach-to-grasp skill learning, we would expect a pattern of neural activity to emerge across the motor network that is closely linked to the control of both fine and gross movements. Alternatively, however, coordination may be achieved in a distributed fashion. In this case, we would expect modular patterns of neural activity to emerge that represent the control of fine or gross movements specifically. We hypothesized that monitoring neural activity across the motor network during learning of a multi-effector skill would allow us to distinguish between these possibilities.

Here, we report that effector-specific neural controllers emerge as a coordinated action is learned. We recorded neural activity in primary motor cortex (M1) and dorsolateral striatum (DLS), the primary striatal target of M1, along with forearm muscle activity throughout learning of a reach-to-grasp skill in rats. We observed that coordinated low-frequency activity emerged across M1, DLS, and forearm muscle activity that represented the control of fast and consistent gross movements. Intriguingly, the emerging control of skilled fine movements was independent of this activity, evolved over a longer timescale, and was primarily represented in M1. Consistent with these results, inactivation of DLS preferentially disrupted skilled gross movements. Together, our results indicate that global movement coordination is achieved through emergent modular neural control.

### RESULTS

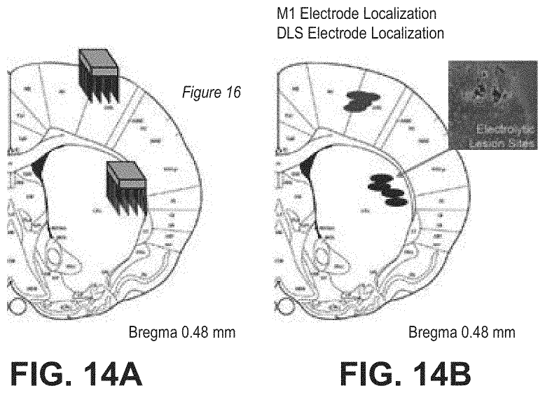

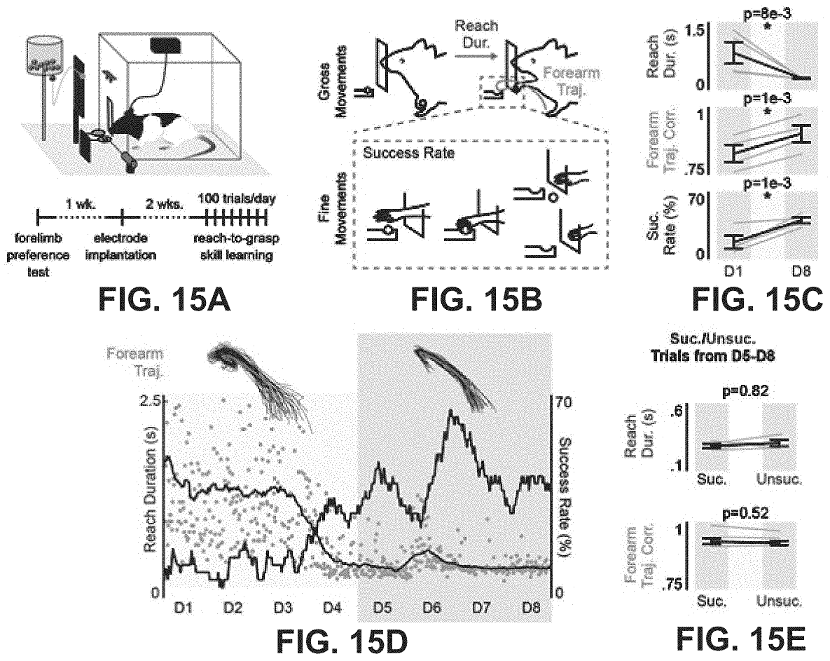

We recorded neural signals, including single-unit activity and local field potentials (LFP) in M1 and DLS (FIG. 14), and forearm muscle activity as rats learned a reach-to-grasp skill. Rats were trained for eight days on the reach-to-grasp skill using automated behavioral boxes, performing 75-100 trials each day (FIG. 15a). Learning this skill requires developing precise control of “gross” movements of forearm, for an accurate reaching action, and “fine” movements of the digits, to successfully grasp the pellet (FIG. 15B). Consistent with past results, training resulted in faster and more consistent movements, as well as increased success rate (FIG. 15c; reach duration: 824±241 ms on day one to 260±8 ms on day eight, mean±SEM hereafter, p=Be-3; forearm trajectory consistency: 0.83±0.03 mean correlation value to 0.90±0.03, p=1e-3; success rate: 25.5±9.7% to 58.0±4.7%, p=1e-3; paired-sample t test, n=4 animals).

Emerging control of skilled fine and gross movements is dissociable. We first sought to determine how changes in success rate were attributable to either changes in fine or gross movements. Intriguingly, we observed that success rate and changes in gross forearm movements, measured by reach duration and forearm trajectory consistency, seemed to evolve on different timescales. While forearm movements stabilized within eight days, success rate remained variable (FIG. 15d, D5-D8). This dissociation suggested that the control of gross movements may stabilize while the control of fine movements of the digits remains variable, resulting in variable success rate. In fact, we observed that differences in forearm movements did not account for success on days five through eight as we found no significant differences between reach duration or forelimb trajectory consistency for successful and unsuccessful trials on these days (FIG. 15e; reach duration: 286±17 ms for successful trials to 308±24 ms for unsuccessful trials, p=0. B2; forearm trajectory correlation: 0.93±0.01 mean correlation value to 0.92±0.01, p=0.52; paired-sample t test, n=4 animals).

Importantly, the control of skilled fine movements continued to evolve on a slower time scale after gross movements stabilized. In a separate “extended training” cohort, performing ˜2500 trials over 4 weeks, average success rate reached a higher rate than our “learning cohort” reached in eight days, while reach duration was not significantly different between cohorts (reach duration: 260±8 ms for learning cohort to 279±45 ms for extended training cohort, p=0.49; success rate: 58.0±4.7% to 78.7±1.1%, p=0.02; unpaired-sample t test, n=4 (training cohort) and 3 (extended cohort) animals). Altogether, this indicated that the emerging control of skilled fine and gross movements was dissociable during reach-to-grasp skill learning.

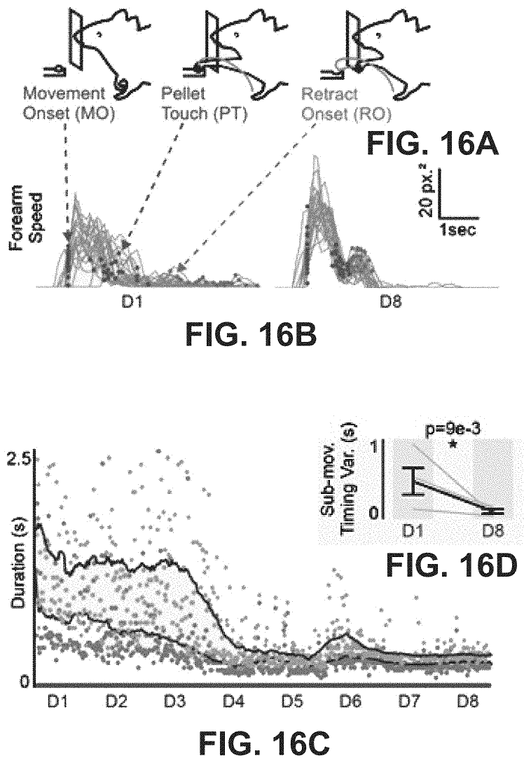

Precise sub-movement timing in skilled gross movements. We next sought to further characterize the emerging control of skilled gross movements. We observed that precise, rhythmic timing of “sub-movements” that make up the reaching action, segmented using the timing of movement onset, pellet touch, and retract onset (FIG. 16a), underlay the faster, more consistent reaches that emerged with training. As reach duration decreased with training, sub-movements became precisely timed and the velocity profile of the forearm developed a consistent multiphasic profile (FIG. 16b-c). This consistency was quantified using the variability of timing between sub-movements, which significantly decreased with training (FIG. 16d; 472±166 ms on day one to 98±29 ms on day eight, p=9e-3, paired-sample t test, n=4 animals).

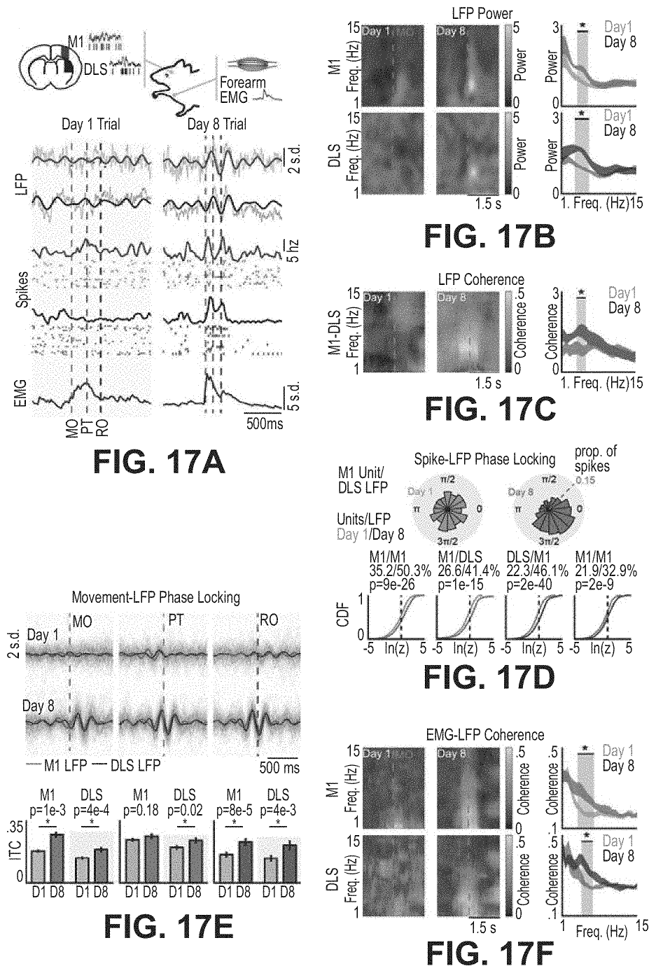

Coordinated low-frequency activity across M1 and DLS represents control of skilled gross movements. We next explored the neural basis for the emerging control of skilled gross movements. Strikingly, we found that rhythmic movement-related neural activity across M1 and DLS reflected the precise rhythmic timing of sub-movements that emerged with training. Specifically, we observed that coordinated low-frequency (˜3-6 Hz) activity emerged during movement across M1 and DLS that was closely related to the timing of sub-movements and forearm muscle activity, which also displayed a similar low-frequency component (FIG. 17a).

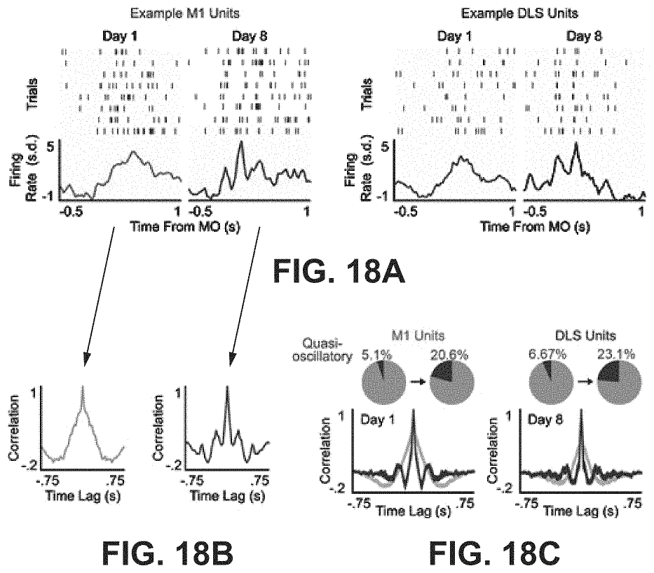

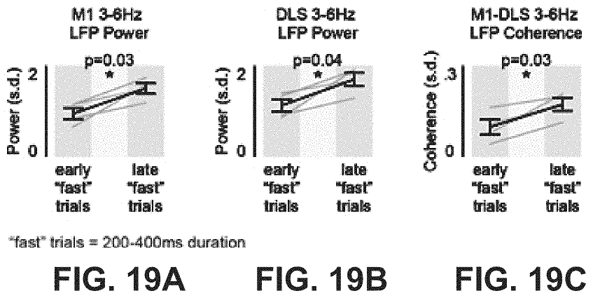

Learning-related changes in movement-related LFP signals and spiking activity consistently displayed the emergence of coordinated low-frequency activity across M1 and DLS. In both M1 and DLS, low-frequency LFP power significantly increased with training (FIG. 17b; M1: significant increase between 4.1-5.3 Hz; DLS: significant increase between 3.5-5.8 Hz; *=p<0.05, paired-sample t test w/Bonferroni correction for multiple comparisons, n=4, hereafter spectrums corresponding to the mean spectrum in each animal). Low-frequency LFP coherence between M1 and DLS also significantly increased with training (FIG. 17c; significant increase between 3.9-5.5 Hz, *=p<0.05, paired-sample t test w/Bonferroni correction for multiple comparisons, n=4 mean spectrums). Phase-locking of M1 and DLS spikes to low-frequency LFP signals also increased with training. Phase-locking was quantified by generating polar histograms of the LFP phases at which each spike occurred for a single unit and LFP channel filtered in the 3-6 Hz band in a one-second window around movement. The non-uniformity of these histograms (indicating phase-locking) was quantified using a Raleigh test of circular non-uniformity that produced a z-statistic with a threshold for significance that allowed us to determine the percentage of unit-LFP pairs that were significantly phase-locked (FIG. 17d; black vertical dotted lines correspond to the p=0.05 significance threshold of the natural log of the z-statistic, all unit-LFP pairs with z-statistics greater than this threshold were significantly phase-locked; M1 unit-M1 LFP pairs: 35.1% day one to 50.3% day eight, p=Be-26, Kolmogorov-Smirnov test, n=2224 pairs on day one and 1696 pairs on day eight; M1 unit-DLS LFP pairs: 26.6% to 41.4%, p=1e-15, Kolmogorov-Smirnov test, n=1536 and 1358 pairs; DLS unit-M1 LFP pairs: 22.3% to 46.1%, p=2e-40, Kolmogorov-Smirnov test, n=1952 and 1264 pairs; DLS unit-DLS LFP pairs: 21.9% to 32.9%, p=3e-9, Kolmogorov-Smirnov test, n=1232 and 784 pairs). The emergence of low-frequency spiking activity was also observed in an LFP-independent manner as the percentage of units that displayed transient oscillatory activity in the 3-6 Hz range during movement increased during learning (FIG. 18). Importantly, the emergence of coordinated low-frequency activity was not solely attributable an increase in movement speed, as coordinated low-frequency activity was not observed during fast movements performed on day one (FIG. 19).

We next characterized the emerging relationship between low-frequency activity across M1 and DLS and both the timing of sub-movements and muscle activity of the forearm. With training, sub-movement timing became precisely phase-locked to the phase of low-frequency activity in both M1 and DLS, consistent with what we would expect if this activity was involved in generating sub-movements (FIG. 17e; significant increase in inter-trial coherence (ITC) of M1 LFP signals locked to movement onset, p=1e-3, and retract onset, p=8e-5, and DLS LFP locked to movement onset, p=3e-4, pellet touch, p=0.02, and retract onset, p=4e-3, paired-sample t test, n=4 mean values from 4 animals; only M1 LFP to pellet touch did not significantly increase in ITC, likely as it was already relatively high on day one). We also observed an increase in low-frequency coherence between forearm muscle activity measured by EMG and LFP signals in both M1 and DLS (FIG. 17f; M1: significant increase between 3.5-7.95 Hz; DLS: significant increase between 4.5-6.9 Hz, *=p<0.05, paired-sample t test w/Bonferroni correction for multiple comparisons, n=4 mean spectrums). Altogether, these results suggested that coordinated low-frequency activity across M1 and DLS represented the emerging control of skilled gross movements.

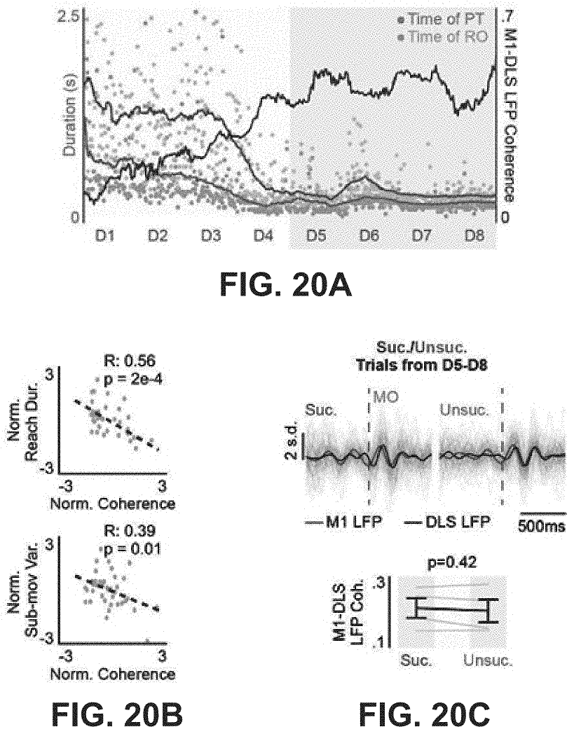

Coordinated M1 and DLS activity is specifically linked to skilled gross, but not fine, movements. If coordinated low-frequency activity across M1 and DLS represented the control of skilled gross movements, we expected their emergence to coincide during learning. In fact, we found that the emergence of movement-related M1-DLS 3-6 Hz LFP coherence closely coincided with the transition to precisely timed sub-movements (FIG. 20a). Across animals, we observed a significant correlation between each session's average movement-related 3-6 Hz M1-DLS LFP coherence and the average reach duration and sub-movement timing variability of that session (FIG. 20b; reach duration: p=2e-4, R=0.56; sub-movement timing variability: p=0.01, R=0.39, Pearson Correlation, n=32 sessions across 4 animals). However, as we observed variable success rate even after the stabilization of gross movements (FIG. 20a, D5-D8), we wondered whether coordinated M1-DLS activity also reflected the variability in success rate. We compared movement-related 3-6 Hz M1-DLS LFP coherence between successful and unsuccessful trials during the period of training after gross movements stabilized and found no significant difference (FIG. 20c; p=0.42, paired-sample t test, n=4 mean values from 4 animals). As we attribute whether trials were successful during this period to the control of skilled fine movements of the digits, this suggested that the control of skilled fine movements was independent of such activity. These results further indicated that emerging coordinated low-frequency activity across M1 and DLS specifically represented the emerging control of skilled gross movements.

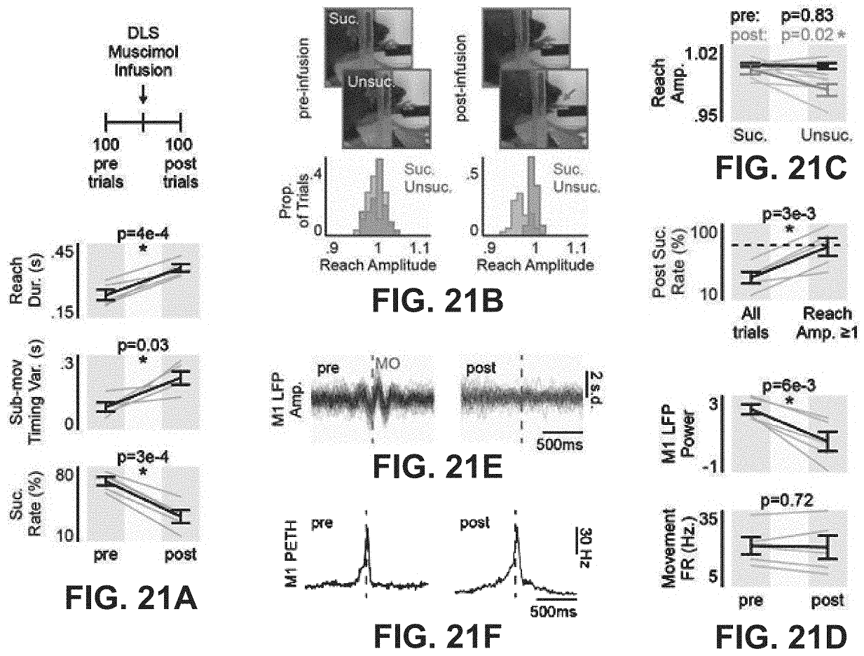

Inactivation of DLS abolishes low-frequency M1 activity and disrupts skilled gross movements. We next sought to further characterize this low-frequency neural activity and its necessity for the control of skilled gross movements, by inactivating DLS with muscimol infusion and observing the effects on skilled movements and M1 activity. In a separate cohort of well-trained animals implanted with infusion cannulas in DLS and electrodes in M1, DLS inactivation significantly impaired reaching performance compared to pre-infusion baseline (FIG. 21a; reach duration: 241.8±20.8 ms to 351.2±14.9 ms, p=4e-4; sub-movement timing variability: 90.9±18.2 ms to 208.2±26.7 ms, p=0.03; success rate: 74.4±4.4% to 36.4±6.9%, p=3e-4, paired-sample t test, n=5 sessions across 3 animals).

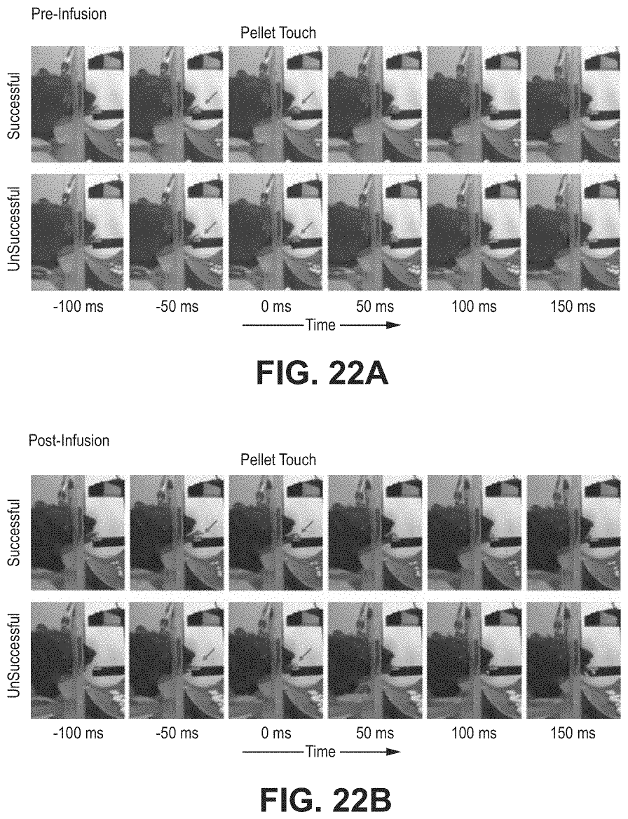

Interestingly, reach amplitude was also decreased after DLS inactivation, consistent with previous work implicating the striatum in movement vigor24,25 (FIG. 21b; FIG. 22). However, we observed a decrease in reach amplitude specifically for unsuccessful trials, but not successful trials after DLS inactivation (FIG. 21c; successful trials: 1.0±0.004, maximum reach amplitude relative to mean maximum amplitude across all trials pre-infusion; unsuccessful: 0.98±0.005, p=0.02, paired-sample t test, n=5 sessions from 3 animals). There was no similar difference in reach amplitude between successful and unsuccessful trials before DLS inactivation (FIG. 21c; successful trials: 1.0±0.001, maximum reach amplitude relative to mean maximum amplitude across all trials pre-infusion; unsuccessful: 1.0±0.002, p=0.B3, paired-sample t test, n=5 sessions from 3 animals). This suggested that decreases in success rate after DLS inactivation may be attributable to impairments in the gross movements involved in transporting the paw to the pellet, rather than a deficit in the fine movements involved in grasping. In fact, success rate for trials after DLS inactivation with a reach amplitude greater than or equal to the average reach amplitude before DLS inactivation was not significantly different than baseline (baseline: 74.4±4.4%, “normal-amplitude” post-infusion trials: 72.6±10.5%, p=0.B9, paired-sample t test, n=5 sessions across 3 animals) and was significantly increased compared to all trials after DLS inactivation (FIG. 21d; all trials: 36.4±6.9%; “normal-amplitude” trials: 72.6±10.5%, p=3e-3, paired-sample t test, n=5 session over 3 animals). This indicated that DLS inactivation preferentially disrupted the control of skilled gross movements involved in the reaching action while leaving the control of skilled fine movements involved in grasping the pellet intact. Infusions of the same volume of saline had no effect on reaching performance compared to pre-infusion baseline (reach duration: 269.4±43.4 ms to 279.2±53.4 ms, p=0.4B; sub-movement timing variability: 100.7±23.5 ms to 82.1±26.7 ms, p=0.07; success rate: 64.6±1.9% to 66.5±7.2%, p=0.B5, paired-sample t test, n=3 sessions over 3 animals).

In M1, there was a significant decrease in movement-related 3-6 Hz LFP power after DLS inactivation compared to pre-infusion baseline (FIG. 21e; p=6e-3, paired-sample t test, n=5 sessions across 3 animals). Intriguingly, this suggested that DLS activity is required for movement-related low-frequency activity in M1. This change was not attributable to a general suppression of M1 activity as we found no significant decrease in movement-related firing rates in M1 after DLS inactivation (FIG. 21f; 21.1±3.4 Hz to 20.5±4.6 Hz, p=0.72, paired-sample t test, n=5 values corresponding to the mean movement-related firing rate across units for 5 sessions across 3 animals). No changes in movement-related LFP power or firing rate were observed after saline infusions compared to pre-infusion baseline (LFP power: p=0.43, paired-sample t test, n=3 sessions across 3 animals; movement-related firing rate: 16.5±6.8 Hz to 17.4±7.8 Hz, p=0.49, paired-sample t test, n=3 values corresponding to the mean firing rates across units for 3 sessions across 3 animals). Altogether we interpreted these results as support for our notion that coordinated low-frequency activity across M1 and DLS represented the control of skilled gross movements and that the control of skilled fine movements was independent of this activity. Intriguingly, it also suggested that the control of skilled fine movements may not rely on DLS activity.

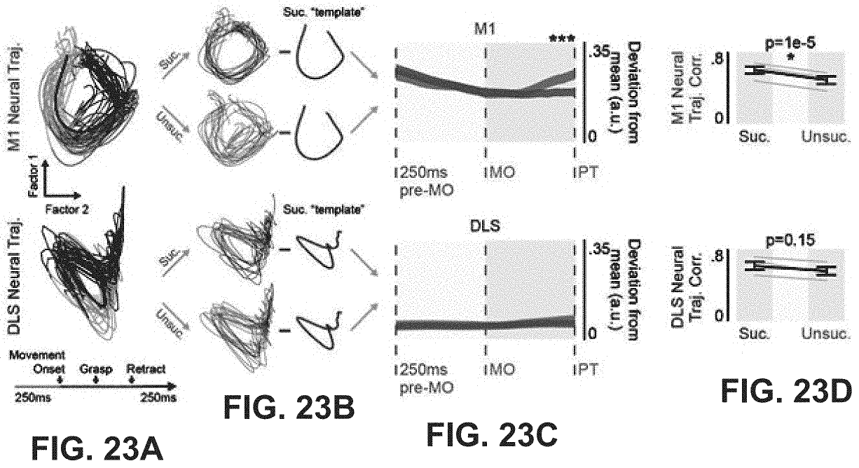

Control of skilled fine movements is represented in M1. Lastly, we sought to investigate whether the control of skilled fine movements was represented in M1 and/or DLS activity. We used gaussian-process factor analysis (GPFA) to find low-dimensional neural trajectory representations of population spiking activity in M1 and DLS on individual trials (FIG. 23a) and then compared trajectories for successful and unsuccessful trials during the period of training after gross movements had stabilized (e.g., FIG. 15d, D5-D8). As we attribute whether trials were successful during this period to the control of skilled fine movements of the digits, we expected to find a difference in movement-related neural signals between successful and unsuccessful trials if a region encodes the control of skilled fine movement. Alternatively, if a region does not encode the control of skilled fine movements, we did not expect to find a difference.

Strikingly, we observed a difference between trajectories for successful and unsuccessful trials in M1 but not DLS. To compare successful and unsuccessful trials we subtracted the mean neural trajectory for successful trials, i.e., the “successful template”, from each individual trial's neural trajectory (FIG. 23b; two dimensions are depicted, but the analysis was performed separately in each of the first four dimensions) and calculated the mean absolute value of the deviation during each time point from 250 ms before movement onset until pellet touch. We focused on this period as it included the fine movements involved in shaping the digits for contact with the pellet but did not include differences in retraction or reward between successful and unsuccessful trials. As trials differed in the duration of this period, we interpolated trajectories during this period such that they were all the same length (see methods). We found that M1 neural trajectories for unsuccessful trials had significantly higher deviation than successful trials starting after movement onset (FIG. 23c, top; *=p<1e-3, unpaired-sample t test w/Bonferroni correction for multiple comparisons, n=570 successful trials and 536 unsuccessful trials across 4 animals). In DLS, however, deviation of successful and unsuccessful trials from the template did not differ (FIG. 23c, bottom), suggesting that the control of skilled fine movements was not represented in DLS. Consistent with this notion, we found a significant increase in mean neural trajectory correlation for successful trials compared to unsuccessful trials in M1 but not DLS (FIG. 23d; M1: 0.60±0.04 mean correlation for successful trials to 0.49±0.04 mean correlation for unsuccessful trials, p=1 e-5; DLS: 0.62±0.05 to 0.56±0.04, p=0.15, paired-sample t test, n=4 mean correlation values across 4 animals). Altogether, this suggested that the control of skilled fine movements was primarily represented in M1, consistent with our finding that DLS inactivation disrupted skilled gross movements while leaving skilled fine movements intact.

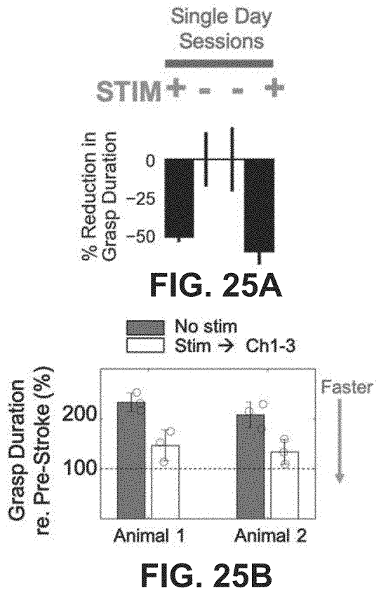

ACS stimulation. In the results shown in FIG. 25, animals were first trained to pick up small objects (e.g. a 8 mm pellet from a deep well). After they achieved stable performance (i.e. time to pellet pick-up was stable over time), a motor cortex stroke was induced. Electrodes were also placed in the epidural space for ACS. As expected, there was a drop in performance, i.e. significant increase in time to manipulate and pick up the object. During periods of time when animals had deficits, we compared performance with and without 3 Hz ACS stimulation. ACS stimulation was applied via low-impedance epidural electrodes in the perilesional cortex relative to a return electrode in the contralateral hemisphere. As shown in FIG. 25A, we saw rapid improvements in performance in the presence of ACS. Without ACS, dexterous performance was significantly worse. A reduction in grasp duration indicated that the animals were able to more rapidly manipulate and pick up the object. In two animals, we consistently observed that animals were improved in their ability to pick up the small pellets. Each dot represents a single session.

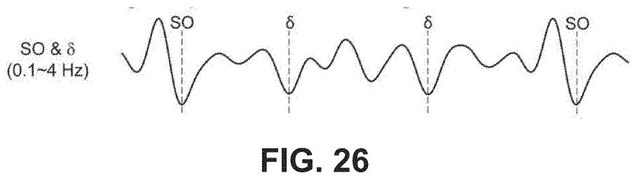

LFO waveforms. FIG. 26 shows the natural variation of LFOs in motor cortex. We also aim to mimic such waveforms in one embodiment of our low-frequency stimulation. As such we will use exponential decay and growth functions to model artificial waveforms that mimic the natural variant (i.e FIG. 1). These waveforms will be used to modulate the current that is delivered.

Our work has found that neural firing in motor cortex can uniquely respond to fluctuations in the field potential. In other words, there are likely to be benefits of customized waveforms that are not simply sinusoidal or biphasic. As shown in FIG. 26, the firing of activity is naturally grouped by an array of natural LFO shapes. For example the larger amplitude events can more easily organize spiking than the lower amplitude waves. Moreover, we have found that the sequence of these waves are essential. This forms the basis of our approach to customizing the waveform shapes with sequences of larger and smaller exponents.

### DISCUSSION

In summary, we found that modular neural control of effectors for “gross” arm and “fine” dexterous movements emerged during reach-to-grasp skill learning in rats. Specifically, coordinated low-frequency activity emerged across M1 and DLS that represented the emerging control of skilled gross movements. Abolishment of this low-frequency activity in M1 by DLS inactivation disrupted the control of skilled gross movements. In contrast, the control of skilled fine movements evolved on a longer time scale, was independent of coordinated low-frequency activity across M1 and DLS, and was not disrupted by DLS inactivation. Consistent with these findings, we found that the control of skilled fine movements was primarily represented in M1.

The neural basis for learning coordinated actions. To our knowledge, this is the first investigation into the emerging neural control of effectors during learning of a coordinated action. Much of the work on motor coordination has focused on forming theoretical frameworks based on behavioral data. A commonly cited framework, based on optimal control theory, posits that movements across effectors are globally optimized to achieve task goals while minimizing parameters such as effort. The current work informs these theories by indicating that such global movement coordination is achieved through the emergence of modular neural controllers. Further work is required to determine whether such modular control generalizes to other forms of coordination (e.g., arm-leg), or is specific to fine and gross effectors. If the latter, this may suggest that distinct neural control is required for effectors that vary greatly in degrees of freedom such as the hand and the arm.

Distributed control of skilled gross movements. Our work indicates that coordinated low-frequency activity across the motor network is essential for the control of skilled gross movements. This is broadly consistent with a growing body of work observing transient oscillatory activity during motor function. In fact, modeling has suggested that low-frequency activity may be an essential feature of neural activity that generates descending commands to muscles. However, past work has exclusively focused on the role of M1 in such a process. Our results suggest that such activity is also present in other nodes in the motor network (i.e. DLS) and, strikingly, that interactions between multiple areas may be required to generate such activity, as we observed a loss of movement-related low-frequency activity in M1 after DLS inactivation. Additional work detailing the precise effect of basal ganglia activity on cortical activity will be central to understanding the role of coordinated activity across cortex and striatum in the control of skilled movements.

Cortical control of skilled fine movements. In contrast to the control of skilled gross movements, we found that the control of skilled fine movements was independent of coordinated low-frequency activity across M1 and DLS and was represented primarily in M1. Intriguingly, this dissociation may indicate a difference in the ability of skilled fine and gross movements to be generated subcortically, suggesting that skilled fine movement may have a greater reliance on cortex. It would be informative to determine whether the observed difference in the emerging neural representations of skilled fine and gross movement control holds for species with significantly greater dexterity, such as non-human primates and humans.

The roles of cortex and striatum in skill learning. In addition to its role in the control of movements, it has been suggested that M1 may provide a “training signal” to allow long-term consolidation of movement sequences into subcortical structures like the DLS, such that M1 is no longer required for movement control14. Our results suggest a neurophysiological substrate for the training signal. For example, it is possible that coordinated low-frequency activity across cortex and striatum provides a mechanism through which M1 activity patterns induce long-term plasticity in the DLS. Modeling has shown that temporally patterned inputs to striatum can drive inter-striatal plasticity31. Further work exploring emerging coordinated activity across the motor network will be essential to understanding the interplay between cortex and striatum, as well as other motor regions such as the cerebellum and thalamus and deeper subcortical structures such as the red nucleus and circuits in the spinal cord, during learning of skilled movements.

### METHODS

### Animal Care and Surgery

All procedures were in accordance with protocols approved by the Institutional Animal Care and Use Committee at the San Francisco Veterans Affairs Medical Center. Animals were kept under controlled temperature and a 12-h light, 12-h dark cycle with lights on at 06:00 A.M. All surgical procedures were performed using sterile technique under 2-4% isoflurane. Surgery involved cleaning and exposure of the skull, preparation of the skull surface (using cyanoacrylate), and then implantation of skull screws for referencing and overall head-stage stability. Reference screws were implanted posterior to lambda, ipsilateral to the neural recordings. Ground screws were implanted posterior to lambda, contralateral to the neural recordings. Craniotomy and durectomy were performed, followed by implantation of neural probes and/or cannulas. Neural probes (32-channel Tucker-Davis Technologies (TDT) 33 μm polyimide-coated tungsten microwire electrode arrays) were implanted in the forelimb area of M1, centered at 3 mm lateral and 0.5 mm anterior to bregma and implanted in layer 5 at a depth of 1.5 mm, and the dorsolateral striatum, centered at 4 mm lateral and 0.5 mm anterior to bregma and implanted at a depth of 5 mm. Cannulas (PlasticsOne) were implanted in the dorsolateral striatum at the same coordinates. Final location of electrodes was confirmed by electrolytic lesion (FIG. 14). The forearm was implanted with a pair of twisted electromyography (EMG) wires (0.007″ single-stranded, teflon-coated, stainless steel wire; A-M Systems, Inc.) with a hardened epoxy ball (J-B Weld) at one end preceded by 1-2 mm of uncoated wire under the ball. Wires were inserted into the muscle belly and pulled through until the ball came to rest on the belly. EMG wires were braided, tunneled under the skin to a scalp incision, and soldered into headstage connectors. Fascia and skin incisions were closed with a suture. The post-operative recovery regimen included administration of buprenorphine at 0.02 mg/kg and meloxicam at 0.2 mg/kg. Dexamethasone at 0.5 mg/kg and Trimethoprim sulfadiazine at 15 mg/kg were also administered post-operatively for 5 days. All animals recovered for 14 days prior to start of behavioral experiments.

### Behavior

For learning experiments, rats naive to any motor training were first tested for forelimb preference. This consisted in presenting approximately ten pellets to the animal and observing which forelimb was most often used to reach for the pellet. One-week later rats underwent surgery followed by a recovery period. Rats were then trained using an automated reach-box, controlled by custom MATLAB scripts and an Arduino micro-controller (FIG. 15a). This setup required minimal user intervention, as described previously. Each trial consisted of a pellet dispensed on the pellet tray followed by an alerting beep indicating that the trial was beginning and then the door opening. Animals had to reach, grasp and retrieve the pellet. A real-time “pellet-detector” using an IR detector centered over the pellet was used to determine when the pellet was moved, indicating the trial was over, and the door was closed. All trials were captured by video, which was synced with electrophysiology data using an Arduino digital output. The training paradigm consisted of 100 trial sessions performed each day for 8 consecutive days. Rats had 15 seconds in each trial to execute a reach before a 10 second inter-trial-interval in which the door was closed, which led to ˜75-100 trials performed (i.e., trials where the pellet was displaced) each day. For the “extended training” cohort, a separate cohort of animals was trained more extensively using the same paradigm for 4 weeks, resulting in ˜2500 trials performed.

### Behavioral Analysis

Learning was assessed using four metrics (FIG. 15b-d): (1) reach duration defined as the time from the onset of movement (movement onset) to when the paw is fully retracted off of the pellet tray (retract onset), (2) sub-movement timing variability defined as the standard deviation across trials of the duration between paw touching the pellet (pellet touch) and when the paw is fully retracted off of the pellet tray (retract onset), (3) success rate defined as the percentage of reaches that resulted in retrieval of the pellet into the box, and (4) forelimb trajectory consistency defined as the average correlation between each individual trial's forelimb trajectory and the mean forelimb trajectory calculated over all trials in that session (computed separately in each of the two dimensions). These metrics were chosen as they measured the relevant changes in both gross movements of the forelimb involved in producing a consistent reach and fine movements of the fingers involved in successful grasping. For the scatter plots comparing changes in reach duration and sub-movement timing variability across learning to changes in movement-related 3-6 Hz M1-DLS LFP coherence (FIG. 20b), normalized values of reach duration and sub-movement timing variability were computed by z-scoring the eight mean values corresponding to the eight days of training for each animal separately, then combining the normalized values across animals.

### Inactivation Experiments

For inactivation experiments, rats were first tested for forelimb preference, then trained for 10 days (100 trials/day) before undergoing cannula and electrode implantation surgery. Following a recovery period, rats began inactivation experiments. For each DLS inactivation experiment, baseline performance was calculated from 100 trials performed before DLS muscimol infusion. Infusion consisted of anesthetizing the rat (w/isoflurane) and infusion of 1 ul of 1 ug/ul muscimol (Tocris) in saline (0.9% sodium chloride) at a rate of 100 nl/min. After the ten-minute infusion and a 5-minute waiting period with the infusion cannula inserted, the rat was taken off anesthesia and allowed to recover for 2 hours. Then another 100 trials block was performed to measure performance during DLS inactivation.

### In Vivo Electrophysiology

Units, LFP, and EMG activity were recorded using a TDT-RZ2 system (Tucker-Davies Technologies). Spike data were sampled at 24414 Hz and LFP/EMG data at 1017 Hz. ZIF-clip-based analog headstages with a unity gain and high impedance (˜1 GO) were used. Behavior-related timestamps (i.e., trial onset, trial completion) and video timestamps (i.e., frame times) were sent to the RZ2 analog input channel using an Arduino digital board and synchronized to neural data.

### Neural Data Analysis

Analyses were conducted using a combination of custom-written scripts and functions in MATLAB 2015a/2017a (MathWorks), along with functions from the EEGLAB toolbox (http://sccn.ucsd.edu/eeglab/) and the Chronux toolbox (http://chronux.org/).

### LFP Analysis

Pre-processing steps for LFP analysis included: artifact rejection (removing broken channels and noisy trials); z-scoring; and common-mode referencing using the median signal (at every time-point, the median signal across all channels in a region was calculated. This median signal was subtracted from every channel to decrease common noise and minimize volume conduction. We used median rather than mean to minimize the effect of channels with high noise. Common-mode referencing was performed independently for the channels in each region, i.e., M1 and DLS).

In several instances we filtered LFP signals to isolate and display the low-frequency (3-6 Hz) component of the signal (FIGS. 17a, e; FIG. 20c; FIG. 21e). Filtering was performed using the EEGLAB function eegfilt. In addition to display purposes, we also used filtered LFP to characterize phase-locking of spiking activity specifically to low-frequency LFP signals. For this we used the Hilbert transform (MATLAB) to extract the phase information from low-frequency filtered LFP signals (FIG. 17e).

To quantify changes across frequencies in the amplitude of rhythmic activity in LFP signals we calculated movement-related LFP spectrograms and power spectrums within each region (FIG. 17b; FIG. 21e). This was carried out using wavelets with the EEGLab function newtimef. To quantify phase-locking of LFP signals to specific sub-movements (movement onset, pellet touch, and retract onset) we calculated inter-trial coherence (ITC) of LFP signals across trials time-locked to these sub-movements (FIG. 17e). ITC was computed using the EEGLab function newtimef.

To characterize coordination of activity across regions we measured changes in movement-related spectral coherence between LFP channels in M1 and DLS (FIG. 17c; FIGS. 20 a, c) or LFP and EMG signals (FIG. 17f). Strong coherence in a specific frequency band indicates a constant phase relationship in that frequency between two signals and is theorized to indicate increased communication between regions. Spectral coherence was computed using chronux function cohgramc. All comparisons of “movement-related” LFP power or coherence used power and coherence values generated from signals between 250 ms before movement onset to 750 ms after movement onset and trial averaging over relevant trials (e.g., all trials on day one or day eight).

To determine whether the emergence of coordinated low-frequency activity during training was attributable solely to faster movements, we compared LFP power and LFP coherence between “fast” trials on days one and two to “fast” trials on days seven and eight. “Fast” trials were characterized by a movement duration between 200 and 400 ms (FIG. 19).

For the scatter plots comparing changes in reach duration and sub-movement timing variability across learning to changes in movement-related 3-6 Hz M1-DLS LFP coherence (FIG. 20b), normalized values of LFP coherence were computed by z-scoring the eight mean values corresponding to the eight days of training for each animal separately, then combining the normalized values across animals.

### Spiking Analysis

Thresholds for spiking activity were set on-line using a standard deviation of 4.5 (calculated over a one-minute baseline period using the TDT-RZ2 system), and waveforms and timestamps were stored for any event that crossed that threshold. Spike sorting was then performed using Plexon OfflineSorter v4.3.0 (Plexon Inc.) with a PCA-based clustering method followed by manual inspection for isolated clusters with clear boundaries. Putative single units were further identified using the following metrics: L-ratio<0.2, Isolation Distance>15, and 99.5% of detected events with ISI>2 ms (acceptable values reported in previous studies). Peri-event time histograms (PETHs) were generated by averaging spiking activity across trials in a session, locked to movement onset and binned at 25 ms (FIG. 21f).

To characterize low-frequency spiking activity, we generated histograms of the LFP phases at which each spike occurred for a single unit to a single LFP channel filtered in the 3-6 Hz band in a one-second window around movement (−250 ms before to 750 ms after movement onset) across all trials of a session (FIG. 17d). These histograms were generated for each unit-LFP channel pair both within and across regions (e.g., if for an example session in one animal we recorded 20 units in M1, 10 units in DLS, and had 16 LFP channels in each region, then we generated 320 histograms for unit-LFP pairs within M1, 160 histograms for unit-LFP pairs within DLS, 320 histograms for M1 unit-DLS LFP pairs, and 160 histograms for DLS unit-M1 LFP pairs). For every pair we then calculated the Rayleigh's z-statistic for circular non-uniformity. These z-statistics were then used to calculate the percentage of significantly non-uniform distributions across unit-LFP pairs with a significance threshold p=0.05 (FIG. 17d). A significantly non-uniform distribution signifies phase preference for spikes of a unit to an LFP signal. To further characterize low-frequency spiking activity, we determined the percentage of units that displayed low-frequency (3-6 Hz) quasi-oscillatory activity. To do this, we computed autocorrelations on each unit's PETH. If a unit's autocorrelation had a “peak” between 166-333 ms time lag (corresponding to 3-6 Hz activity) the unit was considered quasi-oscillatory. A “peak” was defined as a higher average value between 166-333 ms than between 100-166 ms (FIG. 18).

To determine the effects of DLS inactivation on M1 spiking activity we compared movement-related firing rates. Movement-related firing rates were calculated by averaging the firing rate from −250 ms before to 500 ms after movement on each trial of the session (FIG. 21f).

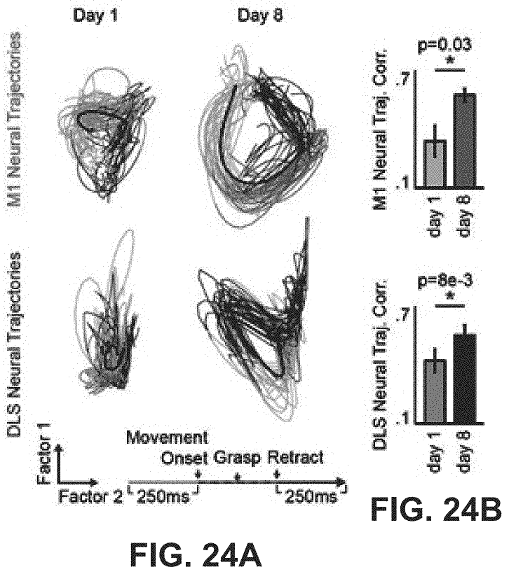

To characterize single-trial representations of population spiking activity we used Gaussian process factor analysis (GPFA) to find low-dimensional neural trajectories for each trial (FIG. 23a; FIG. 24). GPFA analyses were carried out using MATLAB based GUI DataHigh, 25 ms time bins, and a dimensionality of 5. The first four factors were used for analysis as they accounted for >95% of shared variance explained in both M1 and DLS on each session. We found that the consistency of these trajectories, calculated by averaging the correlation of every trial's neural trajectory to the mean neural trajectory of that session (performed in each of the four dimensions or factors) provided a robust measure neural consistency as this measure increased in both M1 and DLS during learning as expected (FIG. 24; M1: p=4e-3; DLS: p=2e-3, paired-sample t test, n=4 mean correlation values from 4 animal; method also used in FIG. 23d). We also determined the magnitude of deviation for each individual trial from the mean trajectory across all successful trials by taking the absolute value of the difference between the trajectory of each trial and the mean trajectory across all trials (FIG. 23b-c; computed in each of the first four dimensions or factors). This was performed specifically for the time period between 250 ms before movement onset until pellet touch. As this duration varied across trials, we interpolated each trial such that every trial was the same length (100 values) then calculated the average deviation.=

[symbol]

### FIGURE LEGENDS

FIG. 2: Changes in Low-Frequency Oscillatory (LFO) Dynamics During Motor Learning. a. Neurophysiological signals were recorded as rats learned a skilled forelimb reach task. b. Average task-evoked power and inter-trial phase-locking (n=4 animals). Red arrow indicates reach onset time in this and all subsequent panels. To quantify significant deflections during the reach period, a paired t-test was performed for each time-frequency point, compared to the mean power for that frequency during a base-line period (−3 to −2 s prior to reach), across trials, followed by FDR-correction (p<0.05). Green shading indicates points that did not reach significance. c. Evolution of LFO power in a single M1 LFP channel over time. White bars separate days (e.g. D1-D8). Z-sc=Z-scored. d. Comparison of LFOs for each submovement for early (Trials #1-25) and late trials (Trials #575-600). Late trials illustrated temporal binding and increased LFO power. e. Significantly increased power (Z-scored) and phase-locking (PLV) from early (first 50) to late (last 50) trials with learning (n=64 recording channels across 4 animals). Stars above indicate specific time points that were significant at a p<0.05, using FDR-corrected paired t-test. f. Example neural spike-field coherence (SFC). Comparison of mean SFC for early and late trials (n=3 animals, 126 neurons), significance assessed using 2-sample t-test. (stars indicate time-points after FDR correction, p<0.05) g. Comparison of LFO PCA trajectories for early and late trials from one animal. Changes in trajectory stereotypy were quantified by calculating the inter-trial trajectory correlation (Fisher-Z transformed) from the first 50 and last 50 trials in each animal, across different 2-Hz band-pass filters (i.e. from 1-3; 2-4; etc.). Stars indicate frequency bands that also show an overall increase (p<0.001, FWE-corrected across 18 frequency bands).

FIG. 3: LFO Dynamics During Motor Recovery After Stroke. a. Focal photothrombotic stroke was performed to induce cortical lesions, followed by implantation of a 16 or 32 channel electrode array in the anterior perilesional cortex. b. Changes in reaching behavior with time (51 was 1 week post stroke for all). Each animal typically attempted 50-75 trials/day. c. Example of change in LFO power with motor recovery. All shown trials involved the animal at least reaching and knocking off the pellet. d. Across animals, there was a significant increase in tasked-related LFO power with time (n=176 channels from 6 animals, paired t-test comparing early and late trials as described in FIG. 2e, *above is 2-sample t-test, p<0.05, FDR-corrected for multiple comparisons across time-points). e. LFO power was a significant predictor of recovery. f. Example change in neural spike-field coherence with recovery (one neuron from first and last sessions). g. Across neurons/animals, we found a significant increase in SFC with recovery (n=296 units). *above is 2-sample t-test, p<0.05, FDR-corrected for multiple comparisons across time-points.

FIG. 4: Modulation of LFO Dynamics Using Direct Current Stimulation (DCS). a. Acute experiments under ketamine anesthesia. Stimulation was performed using a screw implanted posterior to the craniotomy with the ground screw implanted in the contralateral hemisphere. b. Example showing an increase in neural SFC during DCS. c. Example of changes in LFO SFC during DCS (n=7 animals). 46% of neurons showed an increase in excitability (Rate+), 23% of neurons showed a decrease in excitability (Rate−), and 31% of neurons were not modulated (Rate₀). Only those neurons that showed a change in excitability showed a modulation in spike-field coherence (F=13.1, *p<0.001).

FIG. 5: Task-dependent DCS Improves Motor Function. a. Skull-screws for stimulation were implanted both anterior and posterior to the stroke lesion. The ground screw was implanted in the contralateral hemisphere. b. Sessions were pseudo-randomized each day into a block of 30-35 trials. In each block of trials, animals were administered either DC stimulation, a “sham-stim” control (stimulation turned on for only 200 ms), or no stimulation. d. DCS significantly improved reach accuracy after stroke (n=7 animals). There were no significant differences observed in the percent improvement in MCA vs. photothrombotic stroke models.

FIG. 6: Precisely Time-Locked Stimulation Improves Motor Function. a. Stimulation was delivered on every trial pseudo-randomly timed to occur either before, during or after the trial began. The stimulation pulse lasted for only 1 second. ΔT was calculated between the stimulation onset and the actual reach-onset for every trial. b. For each animal, we binned and calculated the percentage accuracy at each ΔT (binning occurred using a window of ±100 ms, with a moving window of 25 ms between time points). We calculated, across animals, the accuracy difference at different ΔTs (accuracy at each ΔT subtracted by the mean accuracy across all trials and stimulation times for that animal). We also found certain time points in which stim was associated with worse performance relative to base-line (denoted in green). Significant improvements denoted by blue diamonds. The corresponding stimulation pulse is denoted in blue above the image. Grey line shows the mean 1.5-4 Hz LFP from healthy animals.

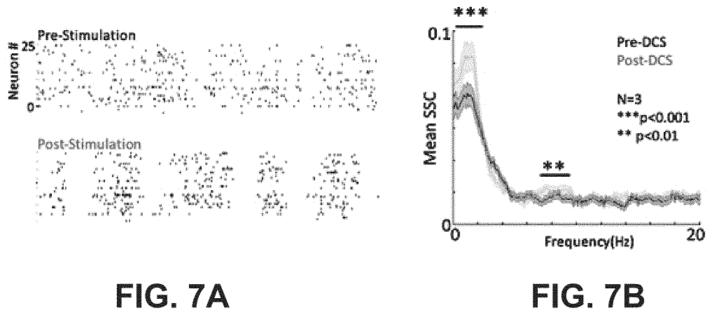

FIG. 7: Enhancement of phase-locking with anodal TDCS during sleep. (A) Example of change in phase-locking with stimulation. Each dot is an action potential. (B) Summary of change in spike-spike coherence (SSC) with simulation. SSC is a measure of how precisely neurons co-fire. Higher values indicate more phase-locking of firing.

FIG. 8: Movement-Related Low-Frequency Oscillations in Sensorimotor Cortex in Humans. a. Center-out paradigm used in patients with ElectroCorticoGraphy (ECoG) recordings. In each trial, subjects were given a hold cue, followed by a “reach” cue that indicated which target to move to. Example of trajectories in the stroke patient. We recorded movement-related data from 2 healthy subjects and 1 stroke subject. Analyses were collapsed across all movement directions in each subject. b. Placement of ECoG grid in the stroke subject, and location of stroke. c. Event-related spectral power across sensorimotor electrodes from one intact subject, and the stroke subject. Power normalized to a base-line time-period for each channel (activity prior to the hold-cue). d. Temporal plot of mean low-frequency power (1.5-4 Hz) from sensorimotor electrodes in each of the 2 intact subjects and the stroke subject. e. Spatiotemporal plot at the 3 time-points indicated in panel (d), demonstrating increase in LF power along the CS (sensorimotor strip) in the two healthy subjects, and absence of this power in the stroke subject.

FIG. 9: Low-frequency quasi-oscillatory (LFO) activity during a skilled forelimb reach task in healthy rats. a. Behavioral setup for skilled forelimb reach task with simultaneous neurophysiological recording. b. Fixed 32-channel micro-wire arrays were implanted in motor cortex. c. Z-scored firing rate changes (171 units from 4 rats) aligned to reach onset. d. Single trial example of brief low-frequency oscillatory activity during reaching (top: spike raster of all units in this example trial, middle: population peri-event time histogram for all spikes shown on top, bottom: z-scored raw LFP in gray and LFP filtered from 1.5-4 Hz in black from an example channel). This trial is representative example of trials that show high SFC and high power, as quantified subsequently. e. Mean spike-field coherence (SFC) across 171 units from 4 rats. f. Mean LFP power across 118 channels from 4 rats. g. 4×8 grid of electrodes from one animal, in actual spatial configuration, with 375 μm spacing in the y-direction and 250 μm spacing in the x-direction, plotting only power from 1.5-6 Hz, and from −0.05 to 0.45 seconds from reach onset.

FIG. 10: Stroke diminished LFO activity in M1. a. Experimental paradigm. After the MCA stroke, we continued recording neural activity from M1 during the reach task in same animals as FIG. 9b. Histological section showing stroke and approximate location of electrodes from one animal. We performed a similar histological analysis in 4 animals to verify that there was some observable lesion resulting from the stroke. c. Pellet retrieval success rate before (mean 48.9%, SD 13.4%) and after (mean 12.4%, SD 13.8%) distal MCA stroke in 4 rats (2-sided paired t-test, t(3)=5.77, *p=0.010). d. Z-scored unit firing rate changes relative to reach onset (53 units from 4 rats). e. Single trial example of diminished LFO activity. Labeling convention is the same as FIG. 9d. Bottom panel shows paw velocity in arbitrary units. This is representative of trials that show low SFC and LFP power, quantified in subsequent panels (g/h) f. Trial-by-trial low frequency LFP power decrease after stroke shown in an example channel, paralleled by decrease in success rate. Left: 1.5-4 Hz LFP power, middle: trial by trial success rate, right: success rate smoothed over 25 trials. Only trials in which rat reached and touched the pellet were included. This is representative of a channel that shows high power prior to stroke and low power after, as quantified in subsequent panels (g/h) g. Quantification of 1.5-4 Hz SFC before (n=171 units) and after (n=53 units) stroke in 4 rats. Thick lines show mean and shaded area is SEM. h. Quantification of changes in low frequency LFP power after stroke, comparing all paired channels (n=101) from all 4 animals. Shaded area is SEM. i. Example grid of channels from the same rat as in FIG. 9 and in the same scale. Channels with spiking activity are enclosed by black squares. Insets 1 and 2 show mean unit waveforms (shaded area is SEM) and inter-spike interval histograms from 2 selected channels. All 4 animals demonstrated a similar loss of low frequency power across channels after the stroke.

FIG. 11: Restoration of LFOs in perilesional motor cortex tracked motor recovery. a. Experimental paradigm. b. Schematic showing location of stroke and electrode. c. Mean pellet retrieval success rate before stroke and during rehabilitation training sessions (n=6, error bars show SEM, grey dots show mean of individual rats). Session 1 or S1 was 1 week post stroke for all. Each animal typically attempted 2 sessions of 25-35 trials each per day. d. Firing rate changes relative to reach onset in early (the first) and late (the last) sessions (for all units from all 6 rats). e. Example of increased LFO activity with rehabilitation, both at the level of spiking and LFP, in two trials with similar paw velocity. Labeling convention are the same as FIG. 10e-f. Example channel from one animal showing trial by trial 1.5-4 Hz LFP power increase, along with success rate increase, over the course of rehabilitation training. Quantification of this effect across channels is in panels i/j. Labeling convention is the same as FIG. 10f. Horizontal white lines separate training sessions. g-h. Mean SFC, calculated from units (n=170 early, n=219 late) in all 6 animals. Shaded area in h is SEM. i-j. Mean LFP power across channels (n=176) from all 6 animals in early and late trials. Shaded area in j is SEM. k. Spatial topography of the low-frequency LFP power increase. Plot shows example channels from one animal. All 6 animals showed similar patterns of recovery, as quantified in panels i/j. I. Scatter showing significant correlation between restoration of low frequency power (mean 1.5-4 Hz power from −0.25 to 0.75 s around reach onset) and improvements on the motor task (r=0.576, two-tailed Pearson's correlation, *p=1.18e-7). Each x represents one session from one rat (n=72 sessions), with values normalized for each animal to first session post-stroke.

FIG. 12: LFO activity increased with Direct Current Stimulation (DCS) in acute (anesthetized) recording sessions. a. Recording and stimulation arrangement in acute experiments. b. LFP power before and during DCS shown in one session. Grey shaded area shows 1.5-4 Hz frequency range. Thick lines in blue and red show mean and shaded areas show SEM. Inset shows 1.5-4 Hz power in pre-DCS and during-DCS in all 11 sessions from 10 rats (mean and SEM shown in bar plots with individual values, two-tailed paired t-test, t(10)=−2.493, *p=0.032). c. Spiking activity of the same neurons from a session before and during stimulation, showing increased coherent spiking during DCS. d. Mean SFC (dark red/blue line—conventions as previous) of 50 neurons from 10 rats. Shaded area represents SEM. 1.5-4 Hz SFC (grey shaded area) increased with DCS (one-tailed paired t-test, t(49)=−1.727, *p=0.045).

FIG. 13: Task-dependent DCS improved motor function post-stroke. a. Cranial-screws placement for stimulation in relation to stroke lesion along with the ground screw. b. Pseudo-randomized stimulation design indicating the trial with either DC stimulation, a “sham-stim” control (stimulation turned on for only 200 ms), or no stimulation. c. Effects of DC vs. sham-stim on motor accuracy on the skilled forelimb reach task post-stroke. Bar plots demonstrate mean/SEM % improvement in accuracy, and lines show the effects in each animal (n=7). We performed one-sample, two-sided t-test performed separately for the Stim (t(6)=6.004, ***p=9.6e-4) and Sham (t(6)=−0.77, p=0.47) group, followed by a paired two-sample two-sided t-test to compare the effects between groups (t(6)=4.91, p=0.003)). d. Mean raw LFP trace (bold line, n=70 trials stim off, n=66 trials stim on) from one animal comparing DCS on vs. off; light grey lines show 6 example single trial traces. Dotted line indicates reach onset time. Quantification performed in next panel. e-f. Mean LFP power for all sessions (n=13 stim on, n=11 stim off sessions) across 4 animals. Bold line in f is the mean and the shaded area is SEM. g. Pseudo-randomized stimulation onset design depicting how a 1 s stimulation was triggered in relation to reach onset. ΔT was negative if the stimulation occurred prior to reach onset, and it was positive if stimulation onset occurred after reach onset. h. Percentage accuracy as a function of ΔT (n=4 animals). Shaded area displays SEM. (*indicates significant improvement in accuracy at ΔT between 500-400 ms from the reach onset, t(3)=9.035, *p=0.046, after Bonferroni-Holm correction for 16 different time points). Grey line shows the mean 1.5-4 Hz LFP from healthy animals, taken from FIG. 9.

FIG. 14: Localization of electrodes. a. Illustration of M1 and DLS recording sites. b. Quantification of electrolytic lesion sites marking electrode locations for four learning animals.

FIG. 15: Emerging control of skilled fine and gross movements is dissociable. a. Illustration of automated behavioral box for reach-to-grasp skill learning (top) and learning paradigm (bottom). b. Illustration of movements involved in reach-to-grasp skill c. Differences in reach duration, forelimb trajectory correlation, and success rate, from day one (D1) to day eight (D8) (light gray lines represent individual animals, black line represents mean with SEM hereafter). d. Example time course of learning (lines are averaged over 30 trials; dots represent individual trials). Forearm trajectories are shown from day one and day eight (mean trajectory in yellow). e. Differences in reach duration and forelimb trajectory correlation for successful and unsuccessful trials from days 5-8.

FIG. 16: Precise movement timing in skilled gross movements. a. Illustration of segmentation of “sub-movements” that make up the reaching action. b. Example forearm speed profiles for trials on day one (D1) and day eight (D8) with timing of sub-movements overlaid. c. Time course of changes in timing of sub-movements over training period. d. Differences in sub-movement timing variability from day one to day eight.

FIG. 17: Coordinated low-frequency activity across M1 and DLS represents control of skilled gross movements. a. Example neural signals from M1 and DLS, sub-movement timing, and forearm muscle activity. b. Spectrograms from example M1 and DLS LFP channels (left) and mean LFP power spectrums, across animals (right; width denotes SEM hereafter, *=p of 0.05, w/Bonferroni correction for multiple comparisons). c. Coherograms from example M1-DLS LFP channel pair (left) and mean coherence spectrum, across animals (right). d. Polar histograms of LFP phases at which spikes occurred for single example M1 unit and DLS LFP channel on day one and day eight (top) and cumulative density functions of z-statistics for every unit-LFP pair across and within each region (bottom; vertical dotted lines denote significance threshold of z-statistic at p<0.05, % of respective unit-LFP pairs greater than threshold noted, lighter color is day one). e. 3-6 Hz filtered LFP from example M1 and DLS channels time locked to sub-movements, individual trials with mean signal overlaid (top), and changes in inter-trial coherence (ITC; bottom). f. Coherograms for example M1 and DLS LFP channels and EMG activity (left) and mean coherence spectrum, across animals (right).

FIG. 18: Percentage of units displaying quasi-oscillatory activity increases during reach-to-grasp skill learning. a. Spiking activity from example units on day one and day eight from M1 and DLS. b. Autocorrelations calculated from example M1 units on day one and day eight from a. c. Quantification of percentage of units in M1 and DLS on day one and day eight that display quasi-oscillatory activity (top) and mean autocorrelation for all quasi-oscillatory and non-oscillatory units on day one and day eight (bottom).

FIG. 19: Coordinated low-frequency activity is not observed in “fast” trials on day one. a. Changes in movement-related M1 3-6 Hz LFP power for “fast” trials (between 200-400 ms duration) on day one and two (early) and day seven and eight (late). b. Same as a for movement-related DLS 3-6 Hz LFP power. c. Same as a for movement-related M1-DLS 3-6 Hz LFP Coherence.

FIG. 20: Coordinated M1 and DLS activity is specifically linked to skilled gross, but not fine, movements. a. Time course of movement-related 3-6 Hz LFP coherence from example M1-DLS channel pair over training period overlaid with timing of sub-movements. b. Scatterplots of each session's mean movement-related 3-6 Hz M1-DLS LFP coherence and mean reach duration and sub-movement timing variability, each normalized per animal. c. 3-6 Hz filtered LFP signals from example M1 and DLS channels for successful and unsuccessful trials on days 5-8 for example animal, individual trials overlaid with mean signal (top) and difference in average M1-DLS LFP coherence for successful and unsuccessful trials on days 5-8, across animals (bottom).

FIG. 21: Inactivation of DLS abolishes low-frequency M1 activity and disrupts skilled gross movements. a. DLS inactivation paradigm and differences in reach duration, sub-movement timing variability, and success rate between trials before (pre) and after (post) DLS inactivation. Grey lines represent individual sessions and black lines represents mean and SEM of all sessions hereafter. b. Snapshot of reach for example successful and unsuccessful trials before and after DLS inactivation, note decrease in reach amplitude for unsuccessful trial compared to successful trial after DLS inactivation (red arrow; top) and histograms of reach amplitude for successful and unsuccessful trials before and after DLS infusion for example animal (bottom). c. Difference in maximum reach amplitude for successful and unsuccessful trials before (black) and after (yellow) DLS inactivation. d. Difference in success rate after DLS inactivation for all trials compared to trials with a maximum reach amplitude greater or equal to the mean maximum amplitude before DLS inactivation. e. 3-6 Hz filtered LFP from example M1 channel before and after DLS inactivation, individual trials overlaid with mean signal (left) and difference in movement-related 3-6 Hz LFP power in M1 before and after DLS inactivation (right). f. PETH from example M1 unit for trials before and after DLS inactivation (left) and changes in movement-related firing rate before and after DLS inactivation (right).

FIG. 22: Difference in reach amplitude for successful and unsuccessful trials before and after DLS inactivation. a. Snapshots of an example successful and unsuccessful reach before DLS inactivation. Note similar reach amplitude for unsuccessful trial compared to successful trial (red arrows). b. Snapshots of an example successful and unsuccessful reach after DLS inactivation. Note decrease in reach amplitude for unsuccessful trial compared to successful trial (red arrows).

FIG. 23: Control of skilled fine movements is represented in M1. a. GPFA neural trajectories for trials on day eight for M1 (top) and DLS (bottom) from example animal. b. Illustration of method for calculating deviation from the mean successful template for successful and unsuccessful trials. c. Mean deviation in (width depicts SEM; computed separately in each factor) from successful template for successful and unsuccessful trials from 250 ms before movement onset to pellet touch, across animals (*=p<0.05, paired-sample t test w/Bonferroni correction for multiple comparisons). d. Difference in average M1 and DLS neural trajectory correlation for successful and unsuccessful trials.

FIG. 24: Changes in GPFA neural trajectory consistency from day one to day eight. a. GPFA neural trajectories for M1 (top) and DLS (bottom) on day one and day eight from example animal. b. Difference in consistency of GPFA trajectories between day one to day eight in M1 (top) and DLS (bottom), across animals.

FIG. 25: Changes with ACS. Animals were first trained to pick up small objects (e.g. a 8 mm pellet from a deep well). After they achieved stable performance (i.e. time to pellet pick-up was stable over time), a motor cortex stroke was induced. Electrodes were also placed in the epidural space for ACS. During periods of time when animals had deficits, we compared performance with and without 3 Hz ACS stimulation. ACS stimulation was applied via low-impedance epidural electrodes in the perilesional cortex relative to a return electrode in the contralateral hemisphere. As shown in FIG. 25A, rapid improvements in performance were observed in the presence of ACS. Without ACS, dexterous performance was significantly worse. A reduction in grasp duration indicated that the animals were able to more rapidly manipulate and pick up the object. In two animals, we consistently observed that animals were improved in their ability to pick up the small pellets. Each dot represents a single session. A. Lack of carry-over between sessions. B. Effects from Ch1-3 Stim is shown for three session per animal, (p<0.005).

FIG. 26: Filtered LFP illustrating diversity of LFO waveform shapes in motor cortex. The dotted line markers the center of each LFO “wave”. This supports the use of customized waveforms during stimulation.

Although the present invention has been described with reference to preferred embodiments, persons skilled in the art will recognize that changes may be made in form and detail without departing from the spirit and scope of the invention.

1. A method for promoting recovery from a stroke induced loss of motor function in a subject comprising:
a. placing at least one recording electrode in electrical communication in a perilesional region of the subject;
b. placing at least one stimulation electrode in electrical communication with the brain of the subject;
c. recording low frequency oscillations (LFOs) from the perilesional region of the subject; and
d. delivering alternating current stimulation to the brain of the subject.

2. The method of claim 1, wherein the alternating current has a waveform selected from the group consisting of monophasic, biphasic, sinusoidal, and customized shapes created using decay and growth time constants.

3. The method of claim 1, further comprising instructing the subject to perform a motor task and monitoring the performance of the subject on the motor task.

4. The method of claim 3, further comprising increasing the amplitude of the delivered alternating current incrementally to the subject until a change in performance of the motor task is detected.

5. The method of claim 4, further comprising decreasing the amplitude of the alternating current delivered to the subject following the detection of the change in motor task performance.

6. The method of claim 1, wherein current is delivered to the perilesional region of the subject.

7. The method of claim 1, wherein the alternating current is delivered to a sleeping subject.

8. The method of claim 1, wherein the at least one stimulation electrode is disposed for synchronized cortical and subcortical stimulation.

9. The method of claim 1, wherein the alternating current stimulation is delivered in phase with the recorded LFOs.

10. The method of claim 1, wherein the alternating current stimulation is delivered at between about 0.1 and about 1000 Hz.

11. The method of claim 1, wherein the alternating current stimulation is delivered in response to changes in recorded electrical activity, wherein the stimulation is delivered when the change is greater than a predetermined threshold change from a baseline activity.

12. The method of claim 1, wherein the alternating current stimulation is delivered in response to subject task performance.

13. The method of claim 1, wherein the one or more stimulation electrodes is placed in at least one of the subcortical white matter, basal ganglia, brainstem, cerebellum or thalamus of the subject.

14. The method of claim 15, wherein a second stimulation electrode is placed in at least one cortical area.

15. The method of claim 1, wherein the one or more stimulation electrodes is placed in at least one cortical area.

16. The method of claim 15, wherein the cortical area the one or more stimulation electrode is placed in a cortical motor area in frontal and parietal cortex.

17. The method of claim 16, wherein a second stimulation electrode is placed in at least one of the subcortical white matter, basal ganglia, brainstem, cerebellum or thalamus of the subject.

18. The method of claim 1, further comprising recording at least one additional frequency wave selected from the group consisting of beta waves, high-gamma waves, gamma waves, alpha waves, delta waves, theta waves, waves of more than 300 Hz and spiking activity/action potentials from neurons as a means of decoding movement intention.

19. A neurostimulation system for improving recovery in a subject with a brain lesion, the neurostimulation system comprising:
a. an electrode constructed and arranged to record low frequency oscillations; and
b. an operations system,
wherein the electrode and operations system are constructed and arranged to:
i) record muscle movement of the subject; and
ii) deliver current to the brain of the subject upon co-occurrence of perilesional low frequency oscillations and subject muscle movement.
deliver current to the brain of the subject in response to low frequency oscillations in the brain.

20. The neurostimulation system of claim 19, wherein the delivered current is alternating current.
